(19) United States
(12) Patent Application Publication
(10) Pub. No.: US 2007/0112593 A1
(43) Pub. Date: May 17, 2007

(54) Selection system, dispensing system and treatment with a one-a-day combination pill for hypertension, hypercholesterolemia, hypertriglyceridemia and anti-platelet treatment

(75) Inventor: Kantilal K. Daya, Boca Raton, FL (US)

Correspondence Address: ROBERT C. KAIN, JR., 750 SOUTHEAST THIRD AVENUE, SUITE 100, FT LAUDERDALE, FL, 333161153, US

(73) Assignee: Rxcera Pharmaceutical, Inc.

(21) Appl. No.: 11/348,786

(22) Filed: Feb. 7, 2006

Related U.S. Application Data
(60) Provisional application No. 60/742,576, filed on Dec. 6, 2005.
(60) Provisional application No. 60/736,355, filed on Nov. 14, 2005.

(51) Int. Cl. G06Q 10/00; A61K 9/00; G06F 7/00

(52) U.S. Cl. 705/2; 424/400; 700/213

(57) ABSTRACT

The present invention relates to a selection system, a dispensing system and a process using a matrix enabling a physician to select one of the plurality of one-a-day combinatory pills. The system and method uses matrices of a plurality of one-a-day pill formulations (a total of 144-177 combinatory pill formulas, or representative indicia thereof). The matrices enable the physician to pick one selected combinatory pill from the 177 formulations, adjust (titrate) dosages over time and use the matrices as an educational tool to motivate the patient. The use of a single daily pill with various commonly prescribed dosages of preventive medications greatly enhances compliance. The information processing system employs a computer or other electronic device or the physician may use printed substrates showing the matrices. The dispensing system includes a dispensing interface.

This is a regular patent application based upon and claiming the priority of provisional patent application Ser. No. 60/742,576 filed Dec. 6, 2005 and Ser. No. 60/736,355 filed Nov. 14, 2005, now pending.

The present invention relates to a method of selection and a selection system for selecting one of a plurality of one-a-day combination pills for primary and secondary (pre and post diagnosis) treatment of hypertension, hypercholesterolemia, hypertriglyceridemia and anti-platelet treatment, an information processing system therefor, a dispensing system for dispensing the selected formulation of the one-a-day combination pill from said plurality of one-a-day combination pills and a method of treatment using said one-a-day combination pill. The one-a-day pills can also be used to forestall diabetes sequeli.

### BACKGROUND OF THE INVENTION

Modifiable major health risk factors can be controlled with appropriate medicaments. It is well known that three major risk factors—serum cholesterol level, blood pressure, and smoking—increase the incidence of coronary heart disease (CHD) and related end points. Long-term studies have amassed extensive data on the relationships of major coronary-cardiovascular risk factors—particularly serum cholesterol level, blood pressure (BP), and cigarette smoking—with incidence of coronary heart disease (CHD), stroke and cardiovascular disease (CVD) to mortality from these causes. These relationships have been characterized as strong, continuous, graded, consistent, independent, predictive, and etiologically significant for CHD and CVD.

Hypertension is well documented risk factor for coronary artery, cerebrovascular, and renovascular disease. Unfortunately, hypertension remains vastly untreated. High blood pressure is a major risk factor for stroke and heart disease.

Studies have also shown that reducing cholesterol in patients prevents the onset of heart disease in apparently healthy persons. Treatment of relatively high risk men with clearly elevated cholesterol levels significantly reduced risk of heart attack and death from heart disease. Persons with heart disease or deemed these to be at high risk for stroke or heart attack should seriously consider treatment if the LDL cholesterol level is greater than about 130 mg/dl. Reports have indicated that reducing LDL cholesterol and total blood cholesterol can reduce the incidence of coronary heart disease and heart attacks in men at high risk because of significant amounts of plasma cholesterol.

Several other studies have shown that treating abnormal lipid levels will reduce cardiovascular morbidity and mortality.

A patient can reasonably control his or her high blood pressure, abnormal cholesterol, tobacco use, diabetes, obesity, and physical activity. Patients cannot control their age, family history of early heart disease (having a father or brother diagnosed with heart disease before age 55 or having a mother or sister diagnosed before age 65), and strongly imbedded habits, such as smoking.

In the healthcare community, there is consistent agreement that the three modifiable risk factors responsible for the cardiovascular and stroke epidemic in the Western World are: elevated uncontrolled blood pressure; elevated cholesterol, and cigarette smoking.

In the past decade the concept has evolved that the intensity of risk factor management should be adjusted according to the severity of estimated risk. Global risk patient assessment is the estimation of absolute risk based on the summation of risks contributed by each risk factor. Several methods have been used to sum risks. The Framingham, Mass. researchers recently proposed a method in which the continuous relationship between risk-factor intensity and coronary risk is employed. Framingham scoring uses only the “standard” risk factors (smoking, blood pressure, serum cholesterol HDL cholesterol, blood glucose and age). Conditional and predisposing risk factors are not used in the Framingham risk equation because of lack of evidence for a strong, independent contribution to CHD risk prediction. Several of the conditional and predisposing risk factors undoubtedly contribute to the development of CHD. Thus, their detection and even therapeutic modification may be appropriate in some patients.

The past decade has witnessed major strides in the prevention of Coronary Heart Disease (CHD) through modification of its causes. The most dramatic advance has been the demonstration that aggressive medical therapy will substantially reduce the likelihood of recurrent major coronary syndromes in patients with established CHD (secondary prevention).

A similar potential exists for risk reduction in patients without established CHD (primary prevention). However, the risk status of persons without CHD varies greatly, and this variability mandates a range in the intensity of interventions. Effective primary prevention thus requires an assessment of risk to categorize patients for selection of appropriate interventions. The major and independent risk factors for CHD are cigarette smoking of any amount, elevated blood pressure, elevated serum total cholesterol and low-density lipoprotein cholesterol (LDL-C), low serum high-density lipoprotein cholesterol (HDL-C), diabetes mellitus, and advancing age. The quantitative relationship between these risk factors and CHD risk has been elucidated by The Framingham Heart Study and other studies. These studies show that the major risk factors are additive in predictive power. Accordingly the total risk of the person can be estimated by the summing of the risk imparted by each of the major risk factors. Other factors are associated with increased risk for CHD: (a) Predisposing Risk Factors such as obesity, abdominal obesity, physical inactivity, family history of premature coronary heart disease, ethnic characteristics, psychosocial factors; and (b) Conditional Risk Factors, such as elevated serum triglycerides, small LDL particles, elevated serum homocysteine, elevated serum Lipoprotein (a), prothrombotic factors (e.g. Fibrinogen), and inflammatory markers (e.g. C-Reactive protein).

These risk factors are of two types: Predisposing risk factors and Conditional risk factors. Conditional risk factors are associated with increased risk for CHD, although their causative, independent, and quantitative contributions to CHD have not been well documented. Predisposing risk factors are those that worsen the independent risk factors. Two of them—obesity and physical inactivity—are designated major risk factors by the American Heart Association because abdominal obesity is an indicator of insulin resistance. Conditional risk factors are those that have been correlated with CHD risk, but their quantitative relationship to major coronary events remains to be defined adequately in large prospective studies. The predisposing risk factors contribute to the development of the causal and conditional risk factors.

### Compliance—A Behavioral Phenomenon

Compliance is commonly understood and defined as “the extent to which the patient's behavior (in terms of taking medication, following diets, or executing other lifestyle changes) coincides with medical recommendations.” Compliance is sometimes defined as patients doing what health professionals want them to do. Compliance with prescribed therapeutic regimens has been a documented concern to health professionals since the time of Hippocrates. Patient compliance with medical regimens is a behavioral problem of interest because it affects the patients health. If the therapeutic regimen is to be effective, the patient must comply with that regimen. No regimen of medication, diet, or behavioral change will benefit the patient who does not follow it.

The role of compliance with medical regimens as a predictor of health outcomes in chronic diseases conditions such as cerebrovascular disorders and diabetes has been the intense focus of recent research. Increasing patient compliance with treatment regimens may decrease hospitalizations and mortality in patient populations as well as improve the quality of life (QOL). Thus, investigations and research over the past decade has focused on the behavioral phenomenon of compliance.

Many researchers have comprehensively reviewed the existing compliance literature. Variations in compliance rates have been found to range between 10%-85% depending on the population, the definition of compliance used and the medical regimen studied. While findings have varied, poor compliance with prescribed therapy has been identified in the literature as an issue that posed serious problems. Poor compliance has direct negative correlations for the health of the patient, effective use of resources and assessments of the clinical efficacy of the treatment.

Recent noncompliance rates for general health-seeking behaviors and lifestyle modifications is set forth below. The results show low general health-seeking behavior. Not only do patients fail to seek medical attention, they also most likely will not stay in care or comply with follow up appointments in over 50% of the time.

Non-Compliance Behavior Table

| TASKS | RATES |
|---|---|
| Community Screening | 35%-90% |
| Referral After Screening | 50%-65% |
| Staying in Care | 31%-66% |
| Follow-up Appointments | 16%-84% |
| Medications | 31%-58% |
| Diet | 13%-76% |
| Activity | 40%-50% |
| Smoking Cessation | 71%-96% |

Compliance rates have been examined for heart failure patients. The results are summarized below. Findings showed that a majority of patients failed to recall elements of potentially important medical advice. Despite some differences in compliance rates in circumstances in which patients did recall medical advice, those that did recall the advice did not always comply with the advice recalled.

Compliance of Patients with Heart Disease

| | | Noncompliance Rate (%) | |
|---|---|---|---|
| | Recalled MD | | Did Not Recall |
| Tasks | Advice % Total | Recalled Advice | Advice |
| Medications | 97.9 | 8.7 | 66.7 |
| Diet (Low Salt) | 83.6 | 23.6 | 55.8 |
| Activity | 70.8 | 76.4 | 84.5 |
| Smoking Cessation | 76.3 | 90.4 | 60.0 |
| Alcohol Use | 42.1 | 60.0 | 81.8 |

In another study involving African American patients with heart conditions measuring the relationship between medication and dietary compliance with hospital readmissions or heart failure HF decompensation, noncompliance was the leading cause for heart failure decompensation, accounting for 43% of hospital admissions. Non-compliance with medication and diet was as high as 64% and 22%, respectively.

Causative factors were identified in 85.5% of the patients in a further study of German heart failure patients. Non-compliance with the medication regimen was the most common identified factor causing heart failure decompensation in 41.9% of cases. Noncompliance with drugs was found in 23% of patients.

A survey of U.S. residents reported that 42% of hypercholesterolemic patients were aware of their condition, although only 4% were adequately treated and controlled.

High blood pressure (HBP) is among the most prevalent and important risk factors for cardiovascular, cerebrovascular, and renal disease. Effective care and control of HBP cannot be achieved without compliance to treatment regimen. Estimates of controlled Blood Pressure (BP) among identified HBP patients typically range from 20%-30% in the U.S., in large part, because only one half of the individuals diagnosed with hypertension are in treatment and one half of these are not receiving treatment adequate to control BP.

In another critical review, it was found that noncompliance rates with prescribed therapeutic regimen range from 30%-60%, and at least 50% of patients for whom drugs are prescribed fail to receive full benefit through inadequate compliance. The high noncompliance rates in HBP treatment have multiple implications at the individual and societal levels. These rates jeopardize patients' health and well being, result in suboptimal health outcomes, lead to inefficient use of health resources, and incur costly treatment for the complications of untreated or inadequately treated HBP. In spite of the critical role played by compliance and the control of HBP, clinicians are not routinely assessing patients' compliance level and patients rarely volunteer this information to their clinician.

The prior practice involved prescribing multiple pills for a patient to treat multiple health conditions. Medication compliance for multiple pills is poor. Further, many people forget to take or become confused as to which pills must be taken at certain times on certain days. The longer the time span from the doctor's appointment, the greater the failure of medication compliance.

### Objects of the Present Invention

It is an object of the present invention to provide a system for selecting a one-a-day combination pill from a large plurality of combination pills for treating a patient with a combination of medicaments for hypertension, hypercholesterolemia, hypertriglyceridemia and anti-platelet medication over a weekly, monthly or quarterly period of time.

It is another object of the present invention to greatly improve patient compliance by providing the one-a-day combination pill over a period of time and having the physician titrate the dosages (adjust the dosages of each effective therapeutic ingredient) over the period of time based upon the condition of the patient.

It is a further object of the present invention to provide a system for selecting one combination pill from numerous combination pills in a standard format identifying statin, diuretic, and ace inhibitor and a medicament use to lower triglyceride (Tricor) with common abbreviations S-D-A-T.

It is a further object of the present invention to use a color coded matrix showing numerous formulations of one-a-day combination pills to facilitate easy selection by the physician.

It is a further object of the present invention to provide a system for selecting having flip charts or pullout charts with the key subgroups of one-a-day combinatory pills.

It is another object of the present invention to provide an information processing system employing a computer, a personal data assistant or other electronic device (having a memory, a display screen and operator input controls) to enable the physician to select one of the plurality of one-a-day combinatory pills.

It is a further object of the present invention to provide an information system which provides data output for the selected one of a one-a-day combinatory pill.

It is a further object of the present invention to provide an information processing system which exchanges data with a dispensing system to enable the selection of one of the one-a-day combinatory pills from the large plurality of pills listed by the information processing system.

It is a further object of the present invention to provide a dispensing system to enable a healthcare professional to select and fill a pill bottle with one of the plurality of one-a-day combinatory pills.

It is a further object of the present invention to provide a dispensing system for the plurality of one-a-day combinatory pills employing a plurality of storage containers each having one formulation of the one-a-day combinatory pills.

It is another object of the present invention to provide a method of treating a patient with a one-a-day combinatory pill for hypertension, hypercholesterolemia, hypertriglyceridemia and anti-platelet medication.

### SUMMARY OF THE INVENTION

The present invention relates to a selection system enabling the physician or other healthcare provider to select one of the plurality of one-a-day combinatory pills, a dispensing system for these large number of pills, an information processing system enabling the physician or healthcare professional to select and dispense the selective one of the plurality of one-a-day combinatory pills and a method of treatment of a patient with the one-a-day combinatory pill. One system for selecting includes a printed substrate having at least three parts and each subpart having a sub plurality of indicia representing the one-a-day pill formulations and means for moving the sub-parts with respect to each other such as flip charts or pullout plates. The matrices of data (pill formulations) may be color coded. The matrices of data are provided to enable the physician to titrate dosages over weekly, monthly or quarterly periods of time based upon the condition of the patient. The primary matrix has four sub-parts which lists a total of 144 combinatory pill formulas (or representative indicia). Further matrices in the system for selecting show an additional 33 combinatory pill formulas. Therefore, the system for selecting enables the physician to pick one selected combinatory pill from 177 formulations, adjust dosages over time (titration of medication) and enables the physician to use the matrices as an educational tool to motivate the patient. The patient sees his or her dosages drop over time by viewing the matrix. The use of a single daily pill with various commonly prescribed dosages of preventive medications greatly enhances compliance. Most people remember to take a single pill each day. A single pill with known medications with commonly used dosages greatly increases compliance and the overall health of the patient.

The information processing system enables the physician or healthcare provider to select one of the 144 one-a-day combinatory pills employing a computer (or other electronic device) with a memory, display screen and operator input controls. The display screen shows, upon respective operator inputs, the first, second, third and fourth pluralities or groups of combinatory pills (parts of the 144 unit matrix) and an output generator shows a selected one of the plurality of one-a-day combinatory pills based upon the healthcare worker's selection. The information processing system may generate a printed version (script) showing indicia of the selected one-a-day combinatory pill and may be electronically coupled to a dispensing system which dispenses the selected one-a-day combinatory pill ordered by the physician.

The dispensing system for the large plurality of one-a-day combinatory pills includes respective storage containers for each formulation of the one-a-day combinatory pill, each pill and each respective storage container having a unique formulation of commonly prescribed dosages of widely use medicaments. A pill dispensing interface accepts a data input from an operator or an electronic data transmission permitting selection of the one-a-day combinatory pill and dispensing from the corresponding container, the selected one of the one-a-day combinatory pill. Dispensing control interfaces use an operator confirmation feedback before the pills are finalized for dispensing.

The method of treating a patient with a one-a-day, orally administered, combinatory pill includes selecting one of the plurality of the combinatory pills and titrating the dosages supplied to the patient after weekly, monthly or quarterly periods of time. The selection of one selected combinatory pill and the change in dosages or titration of dosages of these pills is an important feature of the present invention.

### BRIEF DESCRIPTION OF THE DRAWINGS

Further objects and advantages of the present invention can be found in the detailed description of the preferred embodiments when taken in conjunction with the accompanying drawings in which:

FIG. 1 diagrammatically illustrates an information processing system enabling the healthcare worker to select one of a large plurality of one-a-day combinatory pills, each having different formulations of commonly prescribed medicaments in commonly prescribed or unitized dosages;

FIG. 2 diagrammatically illustrates one embodiment of a dispensing system for the large plurality of one-a-day combinatory pills;

FIG. 3 diagrammatically illustrates another dispensing system for the large plurality of combinatory pills;

FIG. 4 diagrammatically illustrates a different dispensing system for the large plurality of combinatory pills;

FIGS. 5A-5E diagrammatically illustrate a system for selecting one of the plurality of combinatory pills;

FIGS. 6A-6C diagrammatically illustrate a different system for selecting one of the large plurality of combinatory pills; and,

FIG. 7 diagrammatically illustrates another system for selecting one of the large plurality of combinatory pills.

### DETAILED DESCRIPTION OF THE PREFERRED EMBODIMENTS

The present invention relates to a selection system for selecting one of a plurality of one-a-day combination pills for treatment of hypertension, hypercholesterolemia, hypertriglyceridemia and anti-platelet treatment, an information processing system therefor, a dispensing system for dispensing the selected one-a-day combination pill from said plurality of one-a-day combination pills and a method of treatment using said one-a-day combination pill.

The treatment protocol set forth herein is aimed at reducing the incidence of cerebrovascular and cardiovascular diseases, and diabetic sequelae by primary prevention approaches. However, the therapeutic modality utilized is that of secondary prevention. Preventive efforts target each major therapeutically modifiable risk factor. Any major risk factor, if left untreated for many years, has the potential to produce cardiovascular disease. The treatment protocol assess global risk based on the summation of all major risk factors and utilizes them clinically by (a) identifying and measuring patients at risk and (b) managing risk factors. The protocol identifies and measures patients at risk—patients at high risk and patients at low risk—for immediate attention and intervention of the major modifiable risk factors by secondary treatment protocols. Risk factor management entails: (a) Patient enlightenment and motivation, that is, motivate patients to join and adhere to traditional risk reduction therapies ranging in spectrum from diet modification, lifestyle changes to medical therapy; and (b) Modification of the intensity of risk-reduction therapy based upon the global risk estimate, such as stringent diet, exercise, and titration of medication to maintain the risk factors within the parameters of a low risk state.

The first step in the implementation of the one-a-day combination pill treatment is to identify the risk of the patient to CHD, cerebrovascular disease and diabetic sequelae. There are several clinical protocols which may be employed to determine the risk to the patient. The Framingham Heart Study or Framingham Report defined Low Risk as the risk for CHD at any age that is conferred by any combination of all the following parameters: blood pressure<120/<80 mm Hg., total cholesterol 160 to 199 mg./dL. (or LDL-C 100 to 129 mg/dL) for men and 55 mg/dL for women in a non-smoking person with no diabetes. The Framingham study defines a low-risk state as: serum total cholesterol 160 to 199 mg/dL; LDL-C 100 to 129 mg/dL; HDL-C 45 mg/dL. in men and 55 mg/dL in women, blood pressure<120 mm Hg systolic and <80 mm Hg diastolic, nonsmoker; no diabetes mellitus.

Hypertension is rated as follows:

| | Systolic | | | |
|---|---|---|---|---|
| Category | (mmHg) | | Diastolic | Result |
| Normal | less than 115 | And | less than | Good for you |
| | | | 75 | |
| Prehypertension | 115-139 | Or | 75-89 | Your blood pressure could be a problem. Make |
| | | | | changes in what you eat and drink, be physically |
| | | | | active and lose extra weight. If you also have |
| | | | | diabetes, see your doctor. |
| Hypertension | 140-159 or | Or | 90-99 | You have high blood pressure. Ask your doctor or |
| Stage 1 | higher | | or higher | nurse how to control it. |
| Hypertension | 160 or | Or | 100 or | You have high blood pressure. |
| Stage 2 | higher | | higher | |

Government agencies have recommended the following treatment for hypertension: Stage 1 hypertension: thiazide-type diuretics, ACEI, ARB, BB, CCB; Stage 2 hypertension: 2 drug combination—thiazide-type diuretics and ACEI, ARB, BB or CCB. After this treatment, if the patient is not at blood pressure or BP goal, optimize dosages or add additional drugs until goal BP is reached.

Absolute risk is defined as the probability of developing CHD over a given period of time. A recent Framingham report specifies absolute risk for CHD over the next 10 years. The relative risk is the ratio of the absolute risk of the given patient (or group) to that of low-risk group. Literally, the term relative risk represents the ratio of the incidence in the exposed population divided by the incidence in the unexposed persons. The denominator of the ratio can be either the average risk of the entire population or the risk of a group devoid of risk factors. Both the absolute and relative risk is derived from the recently published risk score sheets.

One methodology for risk estimation calculates builds upon the Framingham study and assigns points for each risk factor. The risk factors are age (in gradations from in 5 year blocks from age 30 and points from −1 to 7 up to age 74); LDL cholesterol; HDL cholesterol; blood pressure (BP), diabetes (male: Y or N, 0 or 2 points or female: Y or N, 0 or 4 points, defined as a fasting plasma glucose level>126 md/dL); smoker (Y or N, 0 or 2 points, defined any smoking in the past month).

Global Risk Assessment Scoring - Risk Assessment by Age Table

| Risk | Male | Female |
|---|---|---|
| <34 | −1 | −9 |
| 35-39 | 0 | −4 |
| 40-44 | 1 | 0 |
| 45-49 | 2 | 3 |
| 50-54 | 3 | 6 |
| 55-59 | 4 | 7 |
| 60-64 | 5 | 8 |
| 65-60 | 6 | 8 |
| >70 | 7 | 8 |

Risk Assessment for Total Cholesterol (mg/dl)

| | | |
|---|---|---|
| <160 | −3 | −2 |
| 169-199 | 0 | 0 |
| 200-239 | 1 | 1 |
| 240-279 | 2 | 2 |
| >280 | 3 | 3 |

Risk Assessment for HDL Cholesterol (mg/dl)

| | | |
|---|---|---|
| <35 | 2 | 5 |
| 35-44 | 1 | 2 |
| 45-49 | 0 | 1 |
| 50-59 | 0 | 0 |
| >60 | −2 | −3 |

Risk Assessment for Systolic Blood Pressure

| | | |
|---|---|---|
| <120 | 0 | −3 |
| 120-129 | 0 | 0 |
| 130-139 | 1 | 1 |
| 140-149 | 2 | 2 |
| >160 | 3 | 3 |

In this protocol, age, LDL cholesterol, HDL cholesterol, blood pressure, diabetes, and smoker points are totaled. A 10 year CHD risk projection can be assigned.

CHD Risk Table (determined CHD risk from point total)

| Point | 10 Yr |
|---|---|
| Total | CHD Risk |
| <−3 | 1% |
| −2 | 2% |
| −1 | 2% |
| 0 | 3% |
| 1 | 4% |
| 2 | 4% |
| 3 | 6% |
| 4 | 7% |
| 5 | 9% |
| 6 | 11% |
| 7 | 14% |
| 8 | 18% |
| 9 | 22% |
| 10 | 27% |
| 11 | 33% |
| 12 | 40% |
| 13 | 47% |
| >14 | >58% |

The individual's CHD risk can be compared to his or her population as follows.

CHD Comparison to Others Table (compared to man of the same age)

| | Average | Low |
|---|---|---|
| Age | 10 Yr CHD | 10 Yr CHD |
| (years) | Risk | Risk |
| 30-34 | 3% | 2% |
| 35-39 | 5% | 3% |
| 40-44 | 7% | 4% |
| 45-49 | 11% | 4% |
| 50-54 | 14% | 6% |
| 55-59 | 16% | 7% |
| 60-64 | 21% | 9% |
| 65-69 | 25% | 11% |
| 70-74 | 30% | 14% |

Overweight or obesity increases the risk of developing high blood pressure. Blood pressure (BP) rises as body weight increases. Studies have shown that losing even 10 pounds can lower the blood pressure and losing weight has the biggest effect on those who are overweight and already have hypertension. The two key measures used to determine factors of overweight or obesity are body mass index, or BMI, and waist circumference. BMI is a measure of weight relative to height. It gives an approximation of total body fat. By motivating the patient to loss weight, his or her blood pressure improves.

The relative and absolute risk estimates for CHD in men as determined for Framingham scoring. The relative risk estimates for each age range are compared with baseline risk conferred by age alone (in the absence of other major risk factors). Relative risk is graded and color-coded to include below average, average and moderately above average and high-risk categories. Distinctions in relative risk are arbitrary. Average risk refers to that observed in the Framingham population. Absolute risk estimates are given in the 2 right hand columns. Absolute risk is expressed as a percentage likelihood of developing CHD per decade. Total CHD risk equates to all forms of clinical CHD, whereas hard CHD includes clinical evidence of myocardial infarction and coronary death. Hard CHD estimates are approximated from the published Framingham Data.

| | Age | | | | | | | | | | |
|---|---|---|---|---|---|---|---|---|---|---|---|
| | 30-34 | 35-39 | 40-44 | 45-49 | 50-54 | 55-59 | 60-64 | 65-69 | 70-74 | | |
| | Low Risk Level | | | | | | | | | Absolute Risk | Absolute Risk |
| Points | (2%) | (3%) | (4%) | (5%) | (6%) | (7%) | (8%) | (10%) | (13%) | Total CHD | Hard CHD |
| 0 | 1.0 (1) | | | | | | | | | 2% | 2% |
| 1 | 1.5 (2) | 1.0 | 1.0 | | | | | | | 3% | 2% |
| 2 | 2.0 (3) | 1.3 (1) | 1.3 | 1.0 | | | | | | 4% | 3% |
| 3 | 2.5 (3) | 1.7 (2) | 1.7 (1) | 1.3 | 1.0 | | | | | 5% | 4% |
| 4 | 3.5 (3) | 2.3 (3) | 2.3 (2) | 1.8 | 1.4 | 1.0 | | | | 7% | 5% |
| 5 | 4.0 (4) | 2.6 (3) | 2.6 (2) | 2.0 (1) | 1.6 | 1.1 | 1.0 | | | 8% | 6% |
| 6 | 5.0 | 3.3 (3) | 3.3 (3) | 2.5 (2) | 2.0 (1) | 1.4 | 1.3 | 1.0 | | 10% | 7% |
| 7 | 6.5 | 4.3 (4) | 4.3 (4) | 3.3 (3) | 2.6 (2) | 1.9 (1) | 1.6 | 1.3 | 1.0 | 13% | 9% |
| 8 | 8.0 | 5.3 | 5.3 | 4.0 (4) | 3.2 (3) | 2.3 (2) | 2.0 (1) | 1.6 | 1.2 | 16% | 13% |
| 9 | 10.0 | 6.7 | 6.7 | 5.0 | 4.0 (4) | 2.9 (3) | 2.5 (2) | 2.0 (1) | 1.5 | 20% | 16% |
| 10 | 12.5 | 8.3 | 8.3 | 6.3 | 5.0 | 3.6 (4) | 3.1 (3) | 2.5 (2) | 1.9 (1) | 25% | 20% |
| 11 | 15.5 | 10.3 | 10.3 | 7.8 | 6.1 | 4.4 | 3.9 (4) | 3.1 (3) | 2.3 (2) | 31% | 25% |
| 12 | 18.5 | 12.3 | 12.3 | 9.3 | 7.4 | 5.2 | 4.6 | 3.7 (4) | 2.8 (3) | 37% | 30% |
| 12 | 22.5 | 15.0 | 15.0 | 11.3 | 9.0 | 6.4 | 5.6 | 4.5 | 5.3 (4) | 45% | 35% |
| >14 | 26.5 | >17.7 | >17.7 | >13.3 | >10.6 | >7.6 | >6.6 | >5.3 | >4.1 | >53% | >45% |

boundary markers denoted in ( ):
1 = below risk;
2 = average risk;
3 = moderately above average risk;
4 = high risk

The Framingham Scoring System takes into account gradations in risk factors when estimating absolute risk. The scoring does not adequately account for severe abnormalities of risk factors, e.g. severe hypercholesterolemia, severe hypertension or cigarette smoking. In these cases, Framingham scores can underestimate absolute risk. This underestimation is particularly evident when only one risk factor is present. Thus, heavy smoking or severe hypercholesterolemiacan lead to premature CHD even when the summed score for the absolute risk is not high. Similarly, the many dangers of prolonged, uncontrolled hypertension are well known. These dangers underscore the need for the consulting physician to exercise subjective clinical acumen to control severe risk factors regardless of absolute short-term risk estimates.

In light of the foregoing, the protocol for selecting which one-a-day combinatory pill formulation should be prescribed to a patient is based upon an initial screening and assessment of a patient. This includes (1) measurement of serum levels of total cholesterol (or LDL-C) and HDL-C and evaluation of cholesterol disorders requires measurement of LDL-C, which is the primary target of cholesterol lowering therapy; and (2) measurement of blood pressure (regardless of whether the patient is taking antihypertensive drugs. The average of several blood pressure measurements is required for an accurate determination of the baseline level.

Other factors such as the patient's age, ECG or EKG abnormalities, ABI tests, B-mode ultrasound of carotid, aorta and femoral arteries, ultrasound of carotid arterial intima and media thickness are also diagnostic tools that may be employed by the physician to determine which of the many one-a-day combination pills should be prescribed.

A patient's age is a powerful indicator of absolute risk, because it reflects the total burden of atherosclerosis that has accumulated; the probability of suffering a major coronary event (unstable angina or myocardial infarction) is correlated with total plaque burden.

ECG or EKG abnormalities, such as abnormalities in the rest ECG, nonspecific ST-segment changes and left ventricular hypertrophy, also carry predictive power and can improve office-based risk assessment.

Noninvasive tests of atherosclerotic Burde Ankle-brachial blood pressure Index (ABI) is a simple diagnostic test for lower-extremity peripheral arterial disease (PAD). It is simply the ratio of blood pressure measured in the arteries at the foot or ankle (dorsalis pedis and posterior tibialis arteries, measured by a hand-held Doppler probe) to the blood pressure measured by traditional blood pressure cuff in the arm (brachial artery). Among well-trained operators, test-retest reliability is excellent and the validity of the test for =50% stenosis in leg arteries is high (90% sensitivity and 98% specificity). In population studies, patients with low ABI have been found to have a considerably higher prevalence of CVD (history of myocardial infarction, coronary artery bypass graft surgery, stroke or stroke surgery, or other measures of clinical CVD such as angina or congestive heart failure) compared to those with normal ABI. Such data confirm that atherosclerosis is a diffuse (i.e., systemic) disease and that an abnormal ABI test (ratio<0.90) suggests significant atherosclerosis in other vascular beds. At least 3 prospective studies have shown a strong predictive role for the ABI for CVD morbidity and mortality prediction in persons with PAD detected by ABI.

Many asymptomatic persons aged 50 and over will have abnormal ABI values. Follow-up studies have shown that abnormal ABI provides incremental coronary and all-CVD risk assessment information, over and above that provided by traditional risk factors. For example, in one study, an abnormal ABI increased relative risk for CVD mortality by nearly 4-fold over standard CV risk factors.

B-mode ultrasound is a relatively inexpensive and safe technique that visualizes the lumen and walls of selected arteries, including carotid, aorta, and femoral. B-mode ultrasound has been validated for measuring intima-media thickness (IMT). Cross-sectional associations between common carotid artery IMT and CVD risk factors have been demonstrated in several studies. Similarly, common carotid IMT has been associated with prevalent CVD in cross-sectional studies. At least 4 published studies show that carotid IMT measurement predicts the presence of CHD and its clinical sequelae.

Noninvasive measurements of the intima and media of the common and internal carotid arteries made with high-resolution ultrasonography may form an important base for risk diagnostic purposes. The incidence of cardiovascular events has been correlated with measurements of carotid-artery intima-media thickness. The relative risk of myocardial infarction or stroke increased with intima-media thickness. The relative risk of myocardial infarction or strike (adjusted for age and sex) for the quintile with the highest thickness as compared with the lowest quintile was 3.87 (95% confidence interval, 2.72 to 5.51). The association between cardiovascular events and intima-media thickness remained significant after adjustment for traditional risk factors, showing increasing risk for each quintile of combined intima-media thickness, from the second quintile (relative risk, 1.54, 95% confidence interval, 1.04 to 2.28), to the third (relative risk 1.84, 9r % confidence interval, 1.26 to 2.67), fourth (relative risk, 2.01; 95% confidence interval, 1.38 to 2.91) and fifth (relative risk, 3.15; 95% confidence interval, 2.19 to 4.52). The results of separate analyses of myocardial infarction and stroke paralleled those for the combined endpoint. The study showed that increases in the thickness of the intima and media of the carotid artery, as measured noninvasive by ultrasonography, are directly associated with an increased risk of myocardial infarction and stroke in older adults without a history of cardiovascular disease.

Prevention of clinical atherosclerotic sequelae (myocardial infarction, stroke and peripheral vascular disease) involves modifying reversible risk factors such as systemic hypertension, dyslipidemia, tobacco smoking, and excess body mass index. Studies have demonstrated that atherosclerosis begins at an early age and progresses in an asymptomatic manner over decades.

Patients at high-risk because of multiple risk factors may require intensive modification of risk factors to maximize risk reduction. These guidelines are currently endorsed or supported by various medical organizations and governmental bodies. The reports advocate adjusting the intensity of risk-factor management to the global risk of the patient. In certain reports, overall risk is estimated by adding the categorical risk factors. They do not use a total risk estimate based on summation of risk factors that have been graded according to risk severity. This latter approach is advocated by the Framingham investigators. Framingham reported that some clinicians believe that the summation of graded risk factors provides advantages over the addition of categorical risk factors. The use of graded risk factors has been recommended in risk-management guidelines developed in Europe.

The are several independent factors that effect the selection and use of the one-a-day combination pill. These are: (a) Diabetes Mellitus (a major risk factor for CHD, both Type I and Type II); (b) Elderly Patients (a prominent feature of the Framingham risk score); (c) Hypertriglyceridemia (elevated serum triglycerides are independent risk factor and elevated triglycerides consequently become a target of therapy independent of LDL lowering); (d) Family History of Premature CHD (imparts an incremental risk at any level of global risk factors); (e) Psychosocial Factors (contribution of personality and socioeconomic factors such as economic standing, evocation, racial background, lifestyle, and personality type, increase CHD risk); and (f) Homocysteine (high serum concentration of homocysteine is associated with increased risk for CHD).

### Treatment of Diabetes

The one-a-day pill may also be used to delay the progress of diabetes because the one-a-day pill controls the primordial risk factors that are the same as those which cause targeted organ disease, such a diabetes, that is the one-a-day pill treats high blood pressure, cholesterol and triglyceride levels. Diabetics often have high blood pressure and high cholesterol and are at increased risk of heart attack and stroke. With the one-a-day combinatory pill, (a) diabetics have a greater medication compliance; (b) are less subject to the “sick patient” syndrome; (c) the cost of treatment should be less due to the reduced cost of medication (rather than take 4-5 medications, a single combinatory pill with all 4-5 medications is more cost effective); and (d) the total healthcare cost for the diabetic patient is reduced since the risk of heart attack and stroke and subsequent treatment therefore is reduced. Since diabetics sometimes take several other medicaments each day, a reduction of 4-5 pills and the substitution of one combinatory pill increases compliance and improves patient health. One combinatory pill also significantly reduces patient error involving taking the wrong pills in the wrong dosages.

### Primary Care Usages and Secondary Care Usage

The one-a-day combinatory pill regime is effective both as a primary care treatment and as a secondary care treatment plan. Primary care is called for before the patient suffers from his or her first heart attack or stroke (prior to diagnosis of the CVD or CHD ailment). The risk factor assessment discussed herein enables the physician to employ the one-a-day combinatory pill as a primary care treatment plan. The doctor and the patient can see improvements by titration of dosages and with the use of the pill matrix discussed below.

Secondary care also involves the use of the one-a-day combinatory pill. After a heart attack or a stroke, the combinatory pill may be used titrate the correct dosages for the patient. After the patient leaves the health care facility in a somewhat stabilized condition, the one-a-day pill is easier to prescribe and its use increases patient compliance. Further, patient memory loss, even on a temporary basis, sometimes reduces medication compliance. The one-a-day pill improves compliance since only a single pill is to be taken. The primary care protocol also prescribes the use of a thrombolitic agent. The FDA has indicated that the thrombolitic agent, in the formulations set forth herein, may be taken 4, 8, 16 hours after a Heart Attack as Secondary care in conventional, conservative therapy. The primary care protocol has also been approved by the FDA in an IND Waiver for the use as a thrombolitic agent in the event of an impending cerebrovascular accident whose etiology is based on a stenosed artery due to the accumulation of plaque. A thrombolitic agent is generally not to be taken unless the target blood vessel is at least 66% occluded.

### The One-A-Day Combination Pill Clinical Protocol Risk Factor Management

Having established the absolute risk of the patient to CHD, cerebrovascular disease and diabetic sequelae, the one-a-day pill protocol addresses the management of these independent modifiable risk factors.

A key aspect of the present invention is to establish a process to assure compliance and titration of medication to maintain the risk factors within the parameters of a low risk state, and the administration of thrombolytics when clinically indicated. Upon establishment of absolute risk and selection of the patient for treatment, the patient is treated aggressively as follows. The patient is interviewed in detail with the objective of gleaning information concerning implementation of behavioral and lifestyle modification changes. i.e. diet, exercise and rest. The physician needs to establish a chemotherapeutic regimen prescriptive to the unique needs of the patient. Each modifiable risk factor, such as: hypertension, hypercholesterolemia, hypertriglyceridemia and the elevated aggregative potential of platelets caused by smoking, is assessed individually and aggressively treated with medical therapy.

Each of these medications in the one-a-day combination pill is aimed at reducing the risk potential of the modifiable risk factors is prescribed by many medical protocols. Improving compliance is a key factor in the use of the present one-a-day combinatory pill regime. To improve the probability of compliance of taking all prescribed medication at a specified time of the day, the one-a-day pill protocol requires medication aimed at treating elevated BP (i.e., ACE INHIBITOR), and medication aimed at treating hypercholestrolemia (i.e., Statin Drug), and an anti-platelet aggregative medication (i.e., Plavix/Aspirin), be combined in one capsule. The one capsule a day encourages compliance and assists in eliminating the notions of “a sick person”.

The patient is evaluated at least monthly for the first three months for purposes of titration of dosage and monitoring of side effects for the medications. Titration of the medication is an essential and integral part managing potentially modifiable risk factors.

Since the risk assessment varies for each patient and the patient's dosage for each element in the combinatory pill changes over time, it is helpful to present to the physician a matrix of available combinatory pills. As noted below, the matrix is quite long and complex. The designating indicia S-D-A-T adjacent the mg dosage also greatly assists the physician in selecting one of the 144, matrix I pill formulations. Upon reaching the physician's expectations of maximal management results based upon further visits over time and titration of dosage, office visits are then prescribed by the physician based upon individual patient histories.

The one-a-day combinatory Pill Matrix (below) is understood by the following example: First, each combinatory pill has either aspirin 81 mg or Plavix 75 mg or both and each unique combinatory pill is designated with an S-D-A-T indicia code such as S10-D25-A10-T160 wherein S=Statin Drugs to lower cholesterol; D=Diuretic—a drug to reduce the water in the patients's body thus lowering his or her blood pressure; A=Ace Inhibitor—a hypertension drug that inhibits substances in the body from producing substances that cause high blood pressure (e.g., the following is a list of the ACE inhibitors that are available in the United States: captopril (Capoten), benazepril (Lotensin), enalapril (Vasotec), lisinopril (Prinivil, Zestril) fosinopril (Monopril), ramipril (Altace), perindopril (Aceon), quinapril (Accupril), moexipril (Univasc), and trandolapril (Mavik); T=Tricor—a drug used to lower triglycerides and, to a small degree, cholesterol. TRICOR (fenofibrate tablets), is a lipid regulating agent available as tablets for oral administration. Each tablet contains 48 mg or 148 mg of fenofibrate. The chemical name for fenofibrate is 2-[4-(4-chlorobenzoyl)phenoxy]-2-methyl-propanoic acid, 1-methylethyl ester.

The number following the letter is the medicament's strength in milligrams. For example, S10-D25-A10-T160 is equivalent to S10 or 10 mg of a statin; D25 is 25 mg of a diuretic; A10 is 10 mg of an Ace Inhibitor; and T160 is 160 mg of Tricor. People need various strengths of these widely prescribed drugs to control their risk factors, cholesterol, hypertension, and triglycerides. Rather that giving a person 4 or 5 pills all the drugs in the strengths appropriate for a patient, the drugs are delivered in one pill. Commonly prescribed dosages of S-D-A-T are employed in the 144 one-a-day pill formulations (matrix I).

The first ingredient is either Aspirin 81 mg or Plavix 75 mg or both. Aspirin is approved in one strength 81 mg and Plavix is approved in one strength 75 mg to keep blood platelets from sticking together. The one-a-day pill always contains either 81 mg of aspirin or 75 mg of Plavix or both. PLAVIX (clopidogrel bisulfate) is an inhibitor of ADP-induced platelet aggregation acting by direct inhibition of adenosine diphosphate (ADP) binding to its receptor and of the subsequent ADP-mediated activation of the glycoprotein GPIIb/IIIa complex. Chemically it is methyl (+)-(S)-a-(2-chlorophenyl)-6,7-dihydrothieno [3,2-c]pyridine-5(4H)-acetate sulfate (1:1).

Ace inhibitors block the formation of chemicals in the body that signal the body to increase blood pressure and increase heart rate in addition to the constriction of blood vessels. Beta blockers lower blood pressure.

To lower cholesterol, a patient takes 10, 20 or 40 mg of a statin. Very few people need 80 mg of a statin to lower blood pressure. A patient might take a diuretic either 25 mg or 50 mg. Very few people would take 80 mg of a diuretic. A diuretic is the first line of defense to lower blood pressure. Diuretics help reduce excess water in blood and body tissue. High volumes of water cause the heart to pump harder, thereby increasing BP. An effective drug to lower blood pressure is an Ace Inhibitor. Patients likely take 10 mg 20 mg or 40 mg of an Ace Inhibitor. About one third of the population has elevated triglycerides. To lower them, most patients take a mid-level strength dose of Tricor 160 mgs or a high strength of Tricor 200 mg and a few take the small dose 67 mg. Tricor reduces triglycerides in the blood by altering blood sugar levels. The strength of each drug is dependent on which drug in the class of drugs is used.

The combinatory pill always has aspirin at 81 mg or Plavix at 75 mg. All of the formulations of statins considered have strengths perhaps 10-20-40 and 80 mg it is most likely the combination pill will have ZOROR or LIPITOR but it is possible to use a pill with SIMVASTATIN. The most likely Ace Inhibitor will be MONOPRIL but others could be used are 10-20-40 also. The diuretic intended is SPIRONOLACTONE at 25, 50 and 100, not 80. HYDROCHLOROTHIAZIDE may be used that comes in 25, 50 only or LASIX that comes in 20, 40, 80. Other branded and generic drugs can be used in the combinatory pill.

The term “commonly prescribed dosages” means those dosages that are customarily used by a wide percentage of the population designated to receive the particular drug. The term “unitized” means that the physician would recognize the typical unit dosage such as a statin at 10, 20, or 40 mg. For example, each combinatory pill formulation has a unitized dosage of the medicament in that the dosage listed “S10” is readily recognizable as a widely prescribed statin 10 mg dosage.

Spironolactone (common brand name Aldactone) is a type of medication called a “potassium sparing” diuretic. Diuretics are used to remove a surplus of fluid from the body's bloodstream or tissues. It also acts as an aldosterone inhibitor (prevents salt retention), and is used to treat advanced heart failure when symptoms persist after other drug therapies are maximized. When it is used in this manner, it is not used as a diuretic to remove extra fluid from the body.

Hydrochlorothiazide is a diuretic and antihypertensive. It is the 3,4-dihydro derivative of chlorothiazide. Its chemical name is 6-chloro-3,4-dihydro-2H-1,2,4-benzothiadiazine-7-sulfonamide 1,1-dioxide. Its empirical formula is C₇H₈ClN₃O₄S₂.

Lasix is a diuretic which is an anthranilic acid derivative. Lasix for oral administration contains furosemide as the active ingredient and the following inactive ingredients: lactose monohydrate NF, magnesium stearate NF, starch NF, talc USP, and colloidal silicon dioxide NF. Chemically, it is 4-chloro-N-furfuryl-5-sulfamoylanthranilic acid. Furosemide is available as white tablets for oral administration in dosage strengths of 20, 40 and 80 mg.

Statin Drug Table (examples)

| Trade Name | Generic Name | Sponsor |
|---|---|---|
| Pravachol | Pravastatin | Bristol-Mers Squibb |
| Mevacor | Lovastatin | Merck |
| Zocor | Simvastatin | Merck |
| Lescol | Fluvastatin | Novartis |
| Lipitor | Atorvastatin | Parke-Davis |
| Baycol | Cerivastatin | Bayer |
| Crestor | Rosuvastatin | Astra-Zeneca |
| Advicor | Lovastatin + extended | Kos Pharmaceutical |
| | Release Niacin | |

### One-A-Day Pill Matrix I

The most prevalent risk factor, Elevated Blood Pressure, is identified by the one-a-day protocol as a “primordial risk factor” and is targeted by the one-a-day pill with the chemotherapeutic management tool of a Diuretic and an ACE inhibitor. This is represented in the matrix from the most prevalent dosage used to the least prevalent dosage used. This is categorized by row in each of the 4 columns.

The next primordial risk factor, elevated cholesterol, is targeted by the use of a Statin and is listed in 3 groups of 6 rows based on the most prevalent dosage used to the least prevalent dosage used.

The third most often treated risk factor, elevated Triglycerides, is listed in order of prevalence from left to right in columns A, B and C. TRICOR is not prescribed in all cases, thus the need for column D.

The least prescribed drug strengths, for Statins, i.e. 80 mg, and Diuretics, also 80 mg, are listed in the orange segment, rows 19 to 36 (matrix range D19-D36-A36-A19). The bottom orange segment appears out of order but ranks use by Blood Pressure and then by most frequent matching Statins.

In the combinatory pill, aspirin is always 81 mg and Plavix is always 75 mg. The facilitate the physician in selecting one of the many combinatory pills, Matrix I uses color coding of groups or pluralities of selected ones of the combinatory pills. The color code employed is: highest use is RED, 2nd highest use is Blue, 3^{rd} highest use is Green, and least used combinatory pills is colored Orange.

Aspirin or Plavix Plus, Statin, Diuretic, Ace Inhibitor, with or Without
Tricor (Matrix Table I)

| | A | B | C | D |
|---|---|---|---|---|
| 1 | S10-D25-A10-T160 | S10-D25-A10-T200 | S10-D25-A10-T67 | S10-D25-A10 |
| 2 | S10-D25-A20-T160 | S10-D25-A20-T200 | S10-D25-A20-T67 | S10-D25-A20 |
| 3 | S10-D25-A40-T160 | S10-D25-A40-T200 | S10-D25-A40-T67 | S10-D25-A40 |
| 4 | S10-D50-A10-T160 | S10-D50-A10-T200 | S10-D50-A10-T67 | S10-D50-A10 |
| 5 | S10-D50-A20-T160 | S10-D50-A20-T200 | S10-D50-A20-T67 | S10-D50-A20 |
| 6 | S10-D50-A40-T160 | S10-D50-A40-T200 | S10-D50-A40-T67 | S10-D50-A40 |
| 7 | S20-D25-A10-T160 | S20-D25-A10-T200 | S20-D25-A10-T67 | S20-D25-A10 |
| 8 | S20-D25-A20-T160 | S20-D25-A20-T200 | S20-D25-A20-T67 | S20-D25-A20 |
| 9 | S20-D25-A40-T160 | S20-D25-A40-T200 | S20-D25-A40-T67 | S20-D25-A40 |
| 10 | S20-D50-A10-T160 | S20-D50-A10-T200 | S20-D50-A10-T67 | S20-D50-A10 |
| 11 | S20-D50-A20-T160 | S20-D50-A20-T200 | S20-D50-A20-T67 | S20-D50-A20 |
| 12 | S20-D50-A40-T160 | S20-D50-A40-T200 | S20-D50-A40-T67 | S20-D50-A40 |
| 13 | S40-D25-A10-T160 | S40-D25-A10-T200 | S40-D25-A10-T67 | S40-D25-A10 |
| 14 | S40-D25-A20-T160 | S40-D25-A20-T200 | S40-D25-A20-T67 | S40-D25-A20 |
| 15 | S40-D25-A40-T160 | S40-D25-A40-T200 | S40-D25-A40-T67 | S40-D25-A40 |
| 16 | S40-D50-A10-T160 | S40-D50-A10-T200 | S40-D50-A10-T67 | S40-D50-A10 |
| 17 | S40-D50-A20-T160 | S40-D50-A20-T200 | S40-D50-A20-T67 | S40-D50-A20 |
| 18 | S40-D50-A40-T160 | S40-D50-A40-T200 | S40-D50-A40-T67 | S40-D50-A40 |
| 19 | S80-D25-A10-T160 | S80-D25-A10-T200 | S80-D25-A10-T67 | S80-D25-A10 |
| 20 | S80-D25-A20-T160 | S80-D25-A20-T200 | S80-D25-A20-T67 | S80-D25-A20 |
| 21 | S80-D25-A40-T160 | S80-D25-A40-T200 | S80-D25-A40-T67 | S80-D25-A40 |
| 22 | S80-D50-A10-T160 | S80-D50-A10-T200 | S80-D50-A10-T67 | S80-D50-A10 |
| 23 | S80-D50-A20-T160 | S80-D50-A20-T200 | S80-D50-A20-T67 | S80-D50-A20 |
| 24 | S80-D50-A40-T160 | S80-D50-A40-T200 | S80-D50-A40-T67 | S80-D50-A40 |
| 25 | S10-D80-A10-T160 | S10-D80-A10-T200 | S10-D80-A10-T67 | S10-D80-A10 |
| 26 | S10-D80-A20-T160 | S10-D80-A20-T200 | S10-D80-A20-T67 | S10-D80-A20 |
| 27 | S10-D80-A40-T160 | S10-D80-A40-T200 | S10-D80-A40-T67 | S10-D80-A40 |
| 28 | S20-D80-A10-T160 | S20-D80-A10-T200 | S20-D80-A10-T67 | S20-D80-A10 |
| 29 | S20-D80-A20-T160 | S20-D80-A20-T200 | S20-D80-A20-T67 | S20-D80-A20 |
| 30 | S20-D80-A40-T160 | S20-D80-A40-T200 | S20-D80-A40-T67 | S20-D80-A40 |
| 31 | S40-D80-A10-T160 | S40-D80-A10-T200 | S40-D80-A10-T67 | S40-D80-A10 |
| 32 | S40-D80-A20-T160 | S40-D80-A20-T200 | S40-D80-A20-T67 | S40-D80-A20 |
| 33 | S40-D80-A40-T160 | S40-D80-A40-T200 | S40-D80-A40-T67 | S40-D80-A40 |
| 34 | S80-D80-A10-T160 | S80-D80-A10-T200 | S80-D80-A10-T67 | S80-D80-A10 |
| 35 | S80-D80-A20-T160 | S80-D80-A20-T200 | S80-D80-A20-T67 | S80-D80-A20 |
| 36 | S80-D80-A40-T160 | S80-D80-A40-T200 | S80-D80-A40-T67 | S80-D80-A40 |

Color code boundary chart: high usage is red A1-B1-B6-A6; blue, second highest usage C12-C12-C12-A12; green 3rd highest usage D1-D18-D18-A-18; orange least used dosages D19-D36-A36-A19

### One-A-Day Pill Matrix II

The most prevalent risk factor, Elevated Blood Pressure, identified by the one-a-day pill protocol as a “primordial risk factor”, is targeted by the “one-a-day” pill with the chemotherapeutic management tool of a an ACE inhibitor without a diuretic. This is for that category of physicians who choose not to use a diuretic for the management of elevated blood pressure. This is represented in the matrix from the most prevalent dosage used to the least prevalent dosage used. This is categorized by row in each of the 3 columns.

The next primordial risk factor, elevated Cholesterol, is targeted by the use of a Statin and is listed in 4 groups of 3 rows based on the most prevalent dosage used to the least prevalent dosage used. The third most often treated risk factor, elevated Triglycerides, is listed in order of prevalence from left to right in columns E, F, and G. The least prescribed drug strengths, for Statins, i.e. 80 mg, and Diuretics, also 80 mg, are listed in the orange segment, rows 46 to 48. The bottom orange segment appears out of order but ranks use by Blood Pressure and then by most frequent matching Statins.

Color code for Matrix II: highest usage is RED, 2^{nd} highest usage is Blue, 3^{rd} highest use is Green, and least used is Orange.

Aspirin or Plavix Plus, Statin, Ace Inhibitor, Tricor (Matrix Table II)

| | E | F | G |
|---|---|---|---|
| 37 | S10-A10-T160 | S10-A10-T200 | S10-A10-T67 |
| 38 | S10-A20-T160 | S10-A20-T200 | S10-A20-T67 |
| 39 | S10-A40-T160 | S10-A40-T200 | S10-A40-T67 |
| 40 | S20-A10-T160 | S20-A10-T200 | S20-A10-T67 |
| 41 | S20-A20-T160 | S20-A20-T200 | S20-A20-T67 |
| 42 | S20-A40-T160 | S20-A40-T200 | S20-A40-T67 |
| 43 | S40-A10-T160 | S40-A10-T200 | S40-A10-T67 |
| 44 | S40-A20-T160 | S40-A20-T200 | S40-A20-T67 |
| 45 | S40-A40-T160 | S40-A40-T200 | S40-A40-T67 |
| 46 | S80-A10-T160 | S80-A10-T200 | S80-A10-T67 |
| 47 | S80-A20-T160 | S80-A20-T200 | S80-A20-T67 |
| 48 | S80-A40-T160 | S80-A40-T200 | S80-A40-T67 |

Color code boundaries: red highest use E37-F37-F39-E39; blue 2nd highest use G37-G37-G42-E42; green 3rd highest use G43-G45-E45-E43; orange least used dosages E46-G46-G48-E48.

### One-A-Day Pill Matrix III

Matrix Three (3) is comprised of Aspirin or Plavix, plus various drug combinations to be considered to capture a wider share of the market at the fringe of the one-a-day pill model. These combinations are currently used and a combination pill could replace existing multiple pill regiments. Matrix Three combinations are for the occurrence of an individual risk factor or combinations of risk factors not commonly occurring.

Elevated Blood Pressure Only Aspirin or Plavix Plus: Diuretic and an
Ace Inhibitor (Matrix Table IIIa)

| | H | I | J |
|---|---|---|---|
| 49 | D25-A10 | D25-A20 | D25-A40 |
| 50 | D50-A10 | D50-A20 | D50-A40 |
| 51 | D80-A10 | D80-A20 | D80-A40 |

Elevated Blood Pressure and Elevated Triglycerides Aspirin or Plavix
plus Diuretic, Ace Inhibitor and Tricor (Matrix Table III b)

| | K | L | M |
|---|---|---|---|
| 52 | D25-A10-T160 | D25-A10-T200 | D25-A10-T67 |
| 53 | D25-A20-T160 | D25-A20-T200 | D25-A20-T67 |
| 54 | D25-A40-T160 | D25-A40-T200 | D25-A40-T67 |
| 55 | D50-A10-T160 | D50-A10-T200 | D50-A10-T67 |
| 56 | D50-A20-T160 | D50-A20-T200 | D50-A20-T67 |
| 57 | D50-A40-T160 | D50-A40-T200 | D50-A40-T67 |
| 58 | D80-A10-T160 | D80-A10-T200 | D80-A10-T67 |
| 59 | D80-A20-T160 | D80-A20-T200 | D80-A20-T67 |
| 60 | D80-A40-T160 | D80-A40-T200 | D80-A40-T67 |

Elevated Blood Pressure and Elevated Triglycerides Aspirin or Plavix
Plus Ace Inhibitor and Tricor (Matrix Table III c)

| | N | O | P |
|---|---|---|---|
| 61 | A10-T160 | A10-T200 | A10-T67 |
| 62 | A20-T160 | A20-T200 | A20-T67 |
| 63 | A40-T160 | A40-T200 | A40-T67 |

Elevated Blood Pressure and Elevated Cholesterol Aspirin or Plavix
Plus Statin and Ace Inhibitor (Matrix Table III d)

| | Q | R | S |
|---|---|---|---|
| 64 | S10-A10 | S10-A20 | S10-A40 |
| 65 | S20-A10 | S20-A20 | S20-A40 |
| 66 | S40-A10 | S40-A20 | S40-A40 |
| 67 | S80-A10 | S80-A20 | S80-A40 |

Elevated Blood Pressure and Elevated Cholesterol Aspirin or Plavix
Plus Statin and Diuretic (Matrix Table III e)

| | T | U | V |
|---|---|---|---|
| 68 | S10-D25 | S10-D50 | S10-D80 |
| 69 | S20-D25 | S20-D50 | S20-D80 |
| 70 | S40-D25 | S40-D50 | S40-D80 |
| 71 | S80-D25 | S80-D50 | S80-D80 |

Elevated Cholesterol and Elevated Triglycerides Aspirin or Plavix
Plus Statin and Tricor (Matrix Table III f)

| | W | X | Y |
|---|---|---|---|
| 72 | S10-T160 | S10-T200 | S10-T67 |
| 73 | S20-T160 | S20-T200 | S20-T67 |
| 74 | S40-T160 | S40-T200 | S40-T67 |
| 75 | S80-T160 | S80-T200 | S80-T67 |

### One-A-Day Pill Matrix IV

Matrix Four (4) is comprised of Aspirin or Plavix, plus various drug combinations to be considered to capture a wider share of the market at the fringe of the one-a-day pill model. These combinations are currently used and a combination pill could replace existing multiple pill regiments. Matrix Four combinations are for the occurrence of combinations of risk factors not commonly occurring or treatment regimens not commonly prescribed in the United States.

Elevated Blood Pressure, Cholesterol and Triglycerides Aspirin
or Plavix Plus: Statin, Diuretic and an Tricor (Matrix Table IV a)

| | AA | BB | CC |
|---|---|---|---|
| 76 | S10-D25-T160 | S10-D25-T200 | S10-D25-T67 |
| 77 | S10-D50-T160 | S10-D50-T200 | S10-D50-T67 |
| 78 | S20-D25-T160 | S20-D25-T200 | S20-D25-T67 |
| 79 | S20-D50-T160 | S20-D50-T200 | S20-D50-T67 |
| 80 | S40-D25-T160 | S40-D25-T200 | S40-D25-T67 |
| 81 | S40-D50-T160 | S40-D50-T200 | S40-D50-T67 |
| 82 | S80-D25-T160 | S80-D25-T200 | S80-D25-T67 |
| 83 | S80-D50-T160 | S80-D50-T200 | S80-D50-T67 |
| 84 | S10-D80-T160 | S10-D80-T200 | S10-D80-T67 |
| 85 | S20-D80-T160 | S20-D80-T200 | S20-D80-T67 |
| 86 | S40-D80-T160 | S40-D80-T200 | S40-D80-T67 |
| 87 | S80-D80-T160 | S80-D80-T200 | S80-D80-T67 |

Elevated Blood Pressure and Elevated Triglycerides Aspirin or Plavix
Plus Diuretic, and Tricor (Matrix Table IV b)

| | DD | EE | FF |
|---|---|---|---|
| 88 | D25-T160 | D25-T200 | D25-T67 |
| 89 | D50-T160 | D50-T200 | D50-T67 |
| 90 | D25-T160 | D25-T200 | D25-T67 |
| 91 | D80-T160 | D80-T200 | D80-T67 |

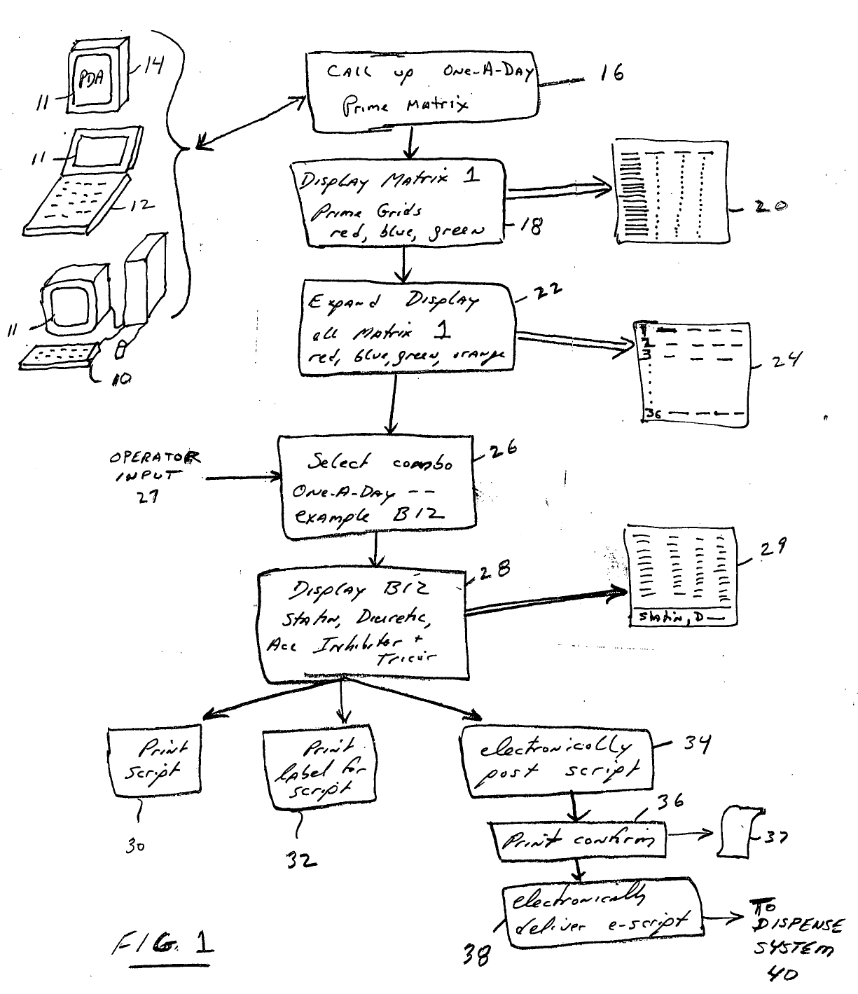

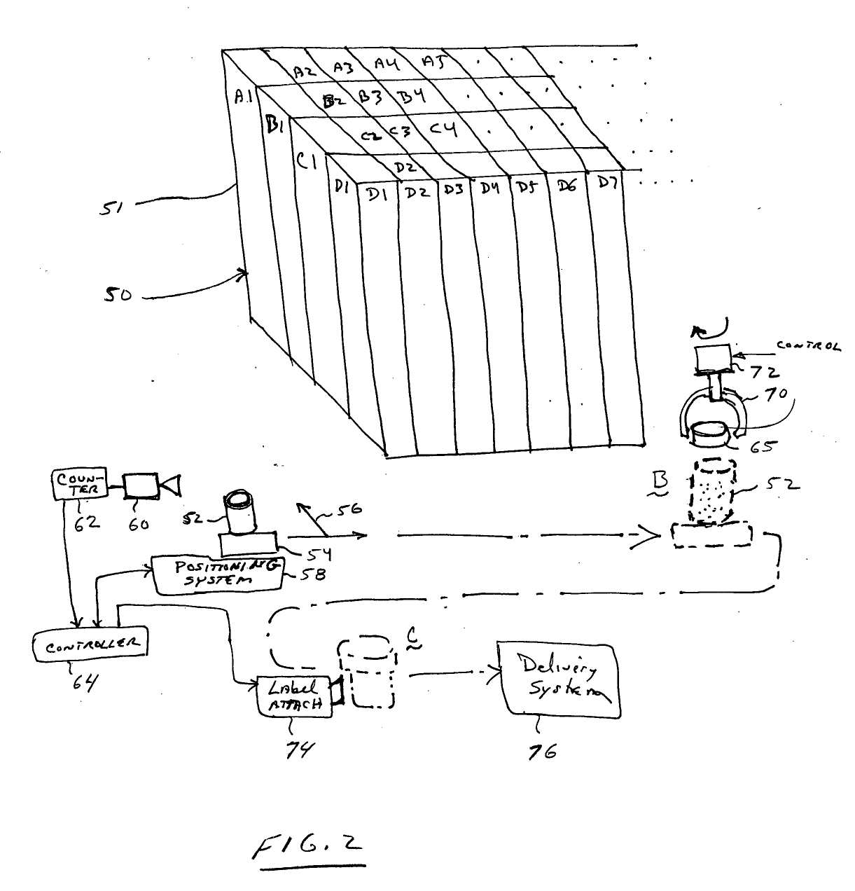

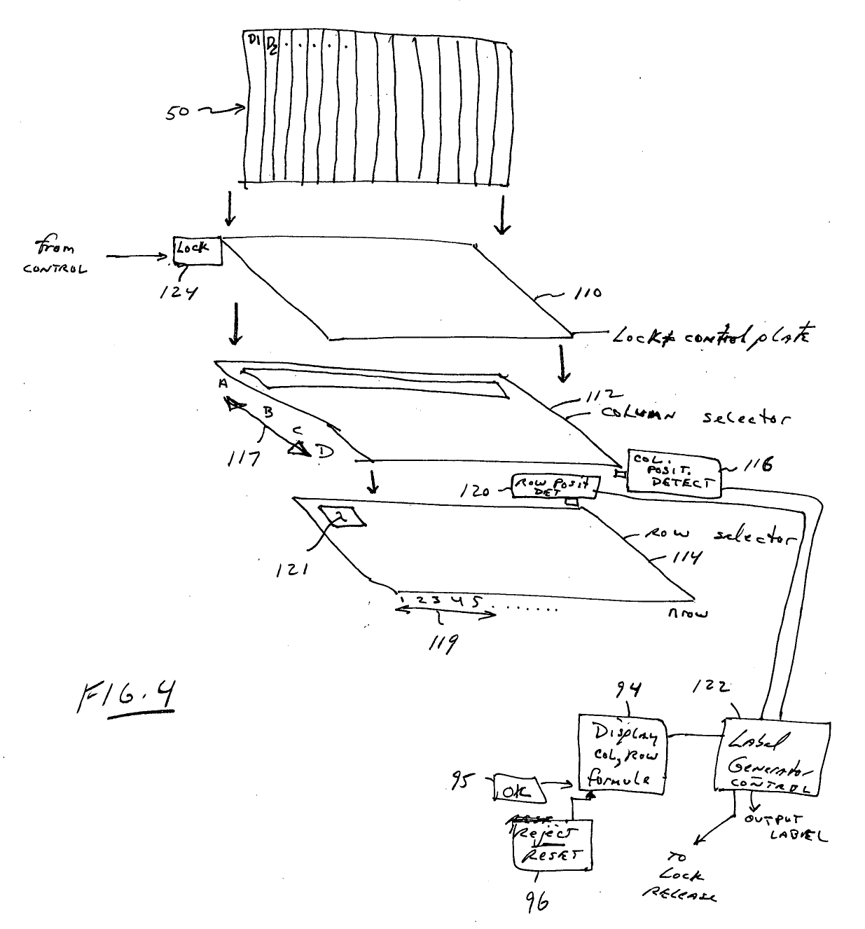

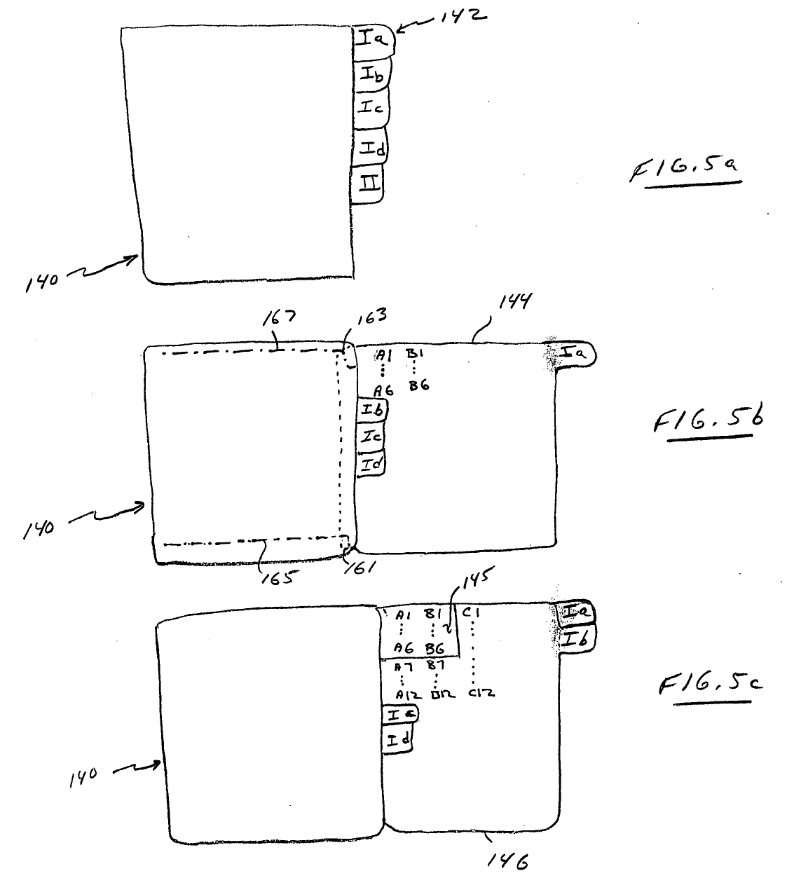

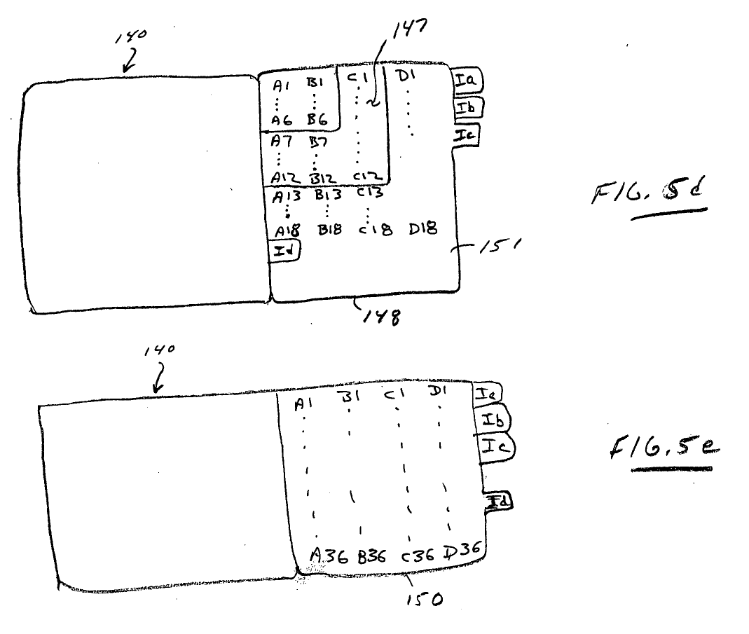

Some of the key elements of the present invention (other key elements are explained herein) involve (a) presenting the data in a compact, easily understood form to the healthcare provider; (b) notifying the healthcare provider that he or she has a wide selection of one-a-day pills of commonly prescribed medicaments in dosages which are commonly prescribed dosages; (c) that these medicaments are delivered in a single pill to be taken by the patient one-a-day; and (d) as result of the one-a-day regimen, the patient compliance significantly increases and the patient is not psychologically impacted with a “sick patient” syndrome. In other words, the one-a-day pill regimen greatly increases compliance and has a significant impact on the long term health of the patient. Previously, the patient would be prescribed 3-4 pills to be taken at various times during the day to improve the patient's health and reduce the patient's risk of CHD and CVD described above. In order to accomplish this selection activity by the healthcare provider, an information processing system is the subject of the present invention (shown in FIG. 1) as is a system for selection of one of the large plurality of one-a-day combinatory pills (shown in FIGS. 5A-5E, 6A-6C and 7). Since the dispensing of a single selected pill from a large plurality of one-a-day combinatory pills is challenging, a dispensing system is diagrammatically illustrated in FIGS. 2-4. Similar numerals designate similar items throughout the drawings.

FIG. 1 diagrammatically shows an information processing system enabling the physician to select one of a large plurality of one-a-day combinatory pills. The information processing system can be employed over any type of computer system as long as that computer system includes a memory, a display screen and operator input controls. Local area networks, wireless networks and Internet applications may be employed. In FIG. 1, a personal computer system 10 is shown, a laptop 12 is shown and a personal data assistant or PDA 14 is shown. These systems all have display screens 11 and include some type of operator input. The keyboard and mouse input on laptop 12 and on personal computer 10 may also include a touch screen input device as is common in PDA 14, display 11. In any event, in functional step 16, the physician or other healthcare provider calls up for display the one-a-day primary matrix. The pill matrices I, II, III and IV are stored in memory. Functional step 18 displays a matrix I (or a primary grid portion thereof) and particularly the primary grids in red, blue and green. The primary grids shown in red, blue and green colors are listed at pill matrix I, column and row A-1, B-1, B-6 and A-6 (grid 1-red); secondary group in blue shown in matrix I at C-1, C-12, A-12 and A-7; and tertiary group without Tricor in matrix I, matrix coordinates D-1, D-18, A-18 and A-13. This is shown diagrammatically in the figure as a matrix with subgroup red, blue and green 20. The display has matrix subgroups red, blue and green (see matrix boundary markers above) which enables the physician to select commonly prescribed medications, in commonly prescribed dosages, in a single pill knowing that patient compliance is higher than if four (4) medications were separately prescribed and dispensed. Also, upon repeat visits, the physician can titrate dosages using the matrix to optimize the patient's health. Further, the healthcare provider can use the matrix with the patient as an educational and motivational tool to motivate the patient to titrate dosages to sub matrix or primary red grid A1, B1, B6, A6.

In functional step 22, the healthcare provider expands the display to include all of combinatory pill matrix I, that is, subgroups red, blue, green and orange. The display is shown in FIG. 1 as display 24 which includes the red grid, blue grid and green grid as well as the orange, least used group of combinatory pills from range A-19, D-19, D-36 and A-36. The information system can quickly shift displays from the primary grid to the secondary, tertiary and full matrix displays for titration purposes and educational purposes.

In functional step 26, the operator has an input (27) which selects one of the plurality of one-a-day combinatory pills such as that listed at grid location B-12. As shown in the matrix I, above, at B-12, in the blue subgroup, the matrix display shows S-20-D50-A40-T200 which represents 20 mg of a statin, 50 mg of diuretic, 40 mg of ace inhibitor and 200 mg of Tricor. The use of the abbreviations S-D-A-T also enable the physician to review many of the combinatory drugs and quickly select one. The indicia S-D-A-T greatly assists in the selection as does the mg dosage next to the letter indicia. In functional step 28, the system, upon operator selection, displays the full formula for the selected one-a-day combinatory drug at grid location B-12. Display screen 29 is shown in FIG. 1. From functional step 28, the information processing system can provide various outputs, one of them being a print script or print prescription function 30, a second being a print label function showing the script as a label but not the entire script prescription pad at functional step 32, and also electronically posting the script at function 34. In function 36 the system prints a confirmation on paper 37 to show the selected one of the plurality of combinatory pills. In step 38 the system electronically delivers the electronic script to a dispensing system 40. Optionally, matrix grid portions maybe printed for reference or to motivate the patient.

With respect to the information processing system, the groupings of sub-pluralities of one-a-day combinatory pill formulations are stored in memory in one of the electronic devices 10, 12 and 14. Of course, a dumb terminal may be utilized rather than a completely independent functional personal computer 10. Further, PDA 14 may be linked via wireless network connection to a main frame or server computer. The output generator is a combination of the touch screen display or the keypad or the mouse in electronic devices 10, 12 and 14 in addition to either a printer output or print script function 30 (also print label 32) or an electronic output as noted in electronic posting of the script in function 34. The output functions 30, 32, 34 may be local or near PDA 14 or may be remote with respect to display screens 11.

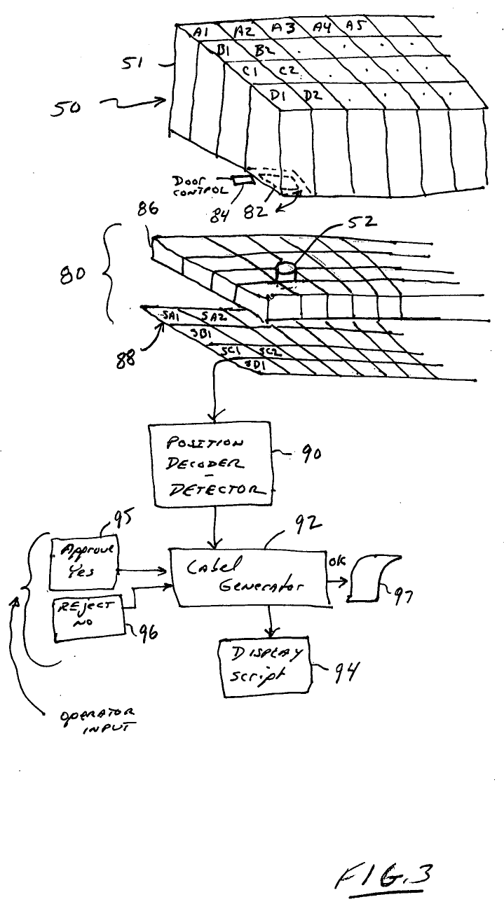

FIG. 2 diagrammatically illustrates a dispensing system as does FIG. 3 and FIG. 4.

With respect to FIG. 2, the dispensing system includes a plurality of containers 50 wherein each respective pill storage container, for example container 51, retains one formulation of the one-a-day combinatory pill. For ease explanation, these containers are marked A1, A2, A3, A4, A5, D1, D2, D3, D4, C1 . . . and B1 . . . . These labels are shorthand designations for the pill combinations in Pill Matrix I and relate to the column and row designators. However, other types of designators may be utilized including the S-D-A-T indicators. If the S-D-A-T indicia are used on the pill and used on storage containers 50, the dispensing operator could easily check and verify pill inventory. Since a large plurality of combinatory pills is available to the consumer, a dispensing system confirming the selected one-a-day pill is an objective of the present invention. Below each respective storage container 51 is a door or a pill dispensing opening with a control latch.

Pill bottle 52 is mounted, in the illustrated embodiment, on positioning trolley 54 which is adapted to move in the x-y direction 56 beneath the plurality of respective storage containers 50. A positioning system controller 58 controls positioner 54 and hence pill bottle 52. A sensing system 60 coupled to a counter detector 52 feeds control pill count signals into controller 64. Controller 64 also provides controlling signal to positioning system 58 thereby moving pill bottle 52 beneath the appropriate respective storage container 51. After the latch or door is open beneath the storage container 51 and pills having a single formulation are dispensed into pill bottle 52 (subject to sensor 60 and counter 62), pill bottle 52 is moved to position B wherein cap 65 is attached to the pill bottle 52. Cap retainer 70 is rotated by drive motor 72 under a control signal from controller 64. Thereafter, pill bottle 52 with cap 65 is moved to position C wherein label attachment system 74 attaches the label that matches the respective storage container 51 established by controller 64. After position C, the pill bottle is delivered to delivery system 76 which ultimately delivers the product to the consumer.

FIG. 3 diagrammatically shows another dispensing system having respective storage containers for each formulation (see plurality of storage containers 50), and a control dispensing interface 80 that differs from the dispensing interface in FIG. 2. The interface in FIG. 2 includes position system 58, positioner 54 and controller 64. It should be noted with respect to both FIG. 2 and FIG. 3, rather than moving the pill bottle 52, the plurality of storage containers 50 may be moved. The important point is that the storage container for the selected one-a-day combinatory pill is placed above or adjacent the pill bottle 52 such that the selected pills as ordered by the healthcare provider are dispensed from the respective container into the empty pill bottle 52. Intermediate hoses or chutes may be interposed between pill bottle 52 and the chosen, selected storage container. The chutes or hoses may be moved rather than the bottle-storage rack.

In FIG. 3, each of the respective storage chambers 50, each having a singular formulation of the plurality of one-a-day pill formulations, includes a dispensing port 82 that is opened or closed via a door or latch under door control 84. Door control 84 is subject to control signals from a controller (not shown) similar to controller 64 in FIG. 2. In FIG. 3, the pill bottle 52 is put in a open top grid structure 86 which has beneath it a plurality of sensors 88. When the pills from storage container C2 (for example) are loaded into pill bottle 52, sensor SC2 is activated thereby indicating that the pills have been dispensed from dispenser storage container C2. Sensor SC2 may also weigh the bottle to sense the condition, empty, partly full or full pill bottle. Position decoding detector 90 decodes the signal from the sensor grid 88. The output from positioned decoder/detector 90 is fed to label generator 92. The label generator is connected to a display unit 94 which shows the storage container which dispensed the pills into pill bottle 52. The operator or pharmacist operating the dispensing system in FIG. 3 should confirm that the displayed formulation on screen 94 conforms to the written prescription related to the patient. The operator then provides an input to approve the pill dispensing or reject the dispensing operation as noted in operator input 95, 96. If rejected, a label is not generated and the pills are not released. If the dispensing process has been approved by the pharmacist or healthcare worker, a label 97 is generated by label generator 92. The label is then attached to pill bottle 52. An automated label attachment sub-system may be incorporated with label generator 92.

FIG. 4 shows another dispensing system which includes a three panel controlled pill dispensing interface consisting of lock and control plate 110, column selector plate 112 and row selector plate 114. In operation, a control unit (similar to control unit 64 in FIG. 2) moves column selector 12 to the appropriate column A, B, C, D (or otherwise) and the position of column selector 112 is detected and recorded by column position detector 116. The opening in column plate 112 is placed beneath the selected storage container. The column selector moves in the direction of double headed arrow 117. Row selector 114 is then moved to the particular row such as row and column D2 and under the selected storage container from the plurality of containers 50. Row selector 114 moves in direction shown in double headed arrow 119. The position of row selector 114 is detected by row position detector 120. When the correct column and row is detected by position detectors 116, 120, these data signals are applied to label generator and control 122. The label generator and control 122 generates a lock release signal which is applied to lock unit 124. Lock unit 124 then releases the lock and control plate 110 and moves the lock and control plate away from the plurality of pill storage containers 50. Otherwise, lock plate may be moved manually away to expose the open row and column. This permits the one selected storage container to dispense pills since a particular row has been selected by row selector 116 and column has been selected by column selector 112. Label generator and control 122 generates a label for the pill bottle which is located beneath space 121 in row selector 114.

As a safety precaution, display unit 94 lists the row and column as well as the selected formula. The healthcare provider must approve or reject the selected row and formula prior to the release of lock 124, the movement of control plate 110 and the production of the label from the label generator control 122.

FIGS. 5A-5E and 6A-6C and 7 show various systems for selecting one of the plurality of one-a-day combinatory pills from various substrates which display various matrices and portions of matrix I. See the red, green, blue and orange subgroups in Matrix I discussed above. In FIG. 5A, booklet 140 has a plurality of tabs 142 which extend from the side, top or bottom. Each tab is labeled with an identifier for the subgroup of combinatory pill matrices such as Matrix Ia which distinguishes the subgroup at Pill Matrix I grid coordinates A-1, B-1, B-6 and A-6. Tab Ib shows the matrix of the first and the second matrix subgroups (red and green) thereby showing the physician grid group A1, C1, C12 and A12. By providing a selection system which lists all the pill combination formulations in a particular subgroups, this greatly assists the physician or healthcare provider to select one of the identified pill formulations and also provides a motivational tool for the patient to move from one lower grid into a higher quality grid such as moving from matrix grid region Ib to grid region Ia. Color coding also provides patient motivation, patient education and dosage titration assistance to the physician. Since titration or the gradual change of medication dosage is contemplated by this invention, grid subgroups and matrix displays are quite helpful to the physician. FIG. 5B shows booklet 140 with a pullout sleeve 144 associated with the tab Ia. The printed subpart substrate shows the primary grid A1, B1, B6 and A6 pill formulations. FIG. 5C shows a second printed subpart substrate with the pill formulation grid pattern C1, C12, A12 and A7 (secondary sub-matrix). A window 145 on printed substrate 146 shows the underlying or primary grid A1, B1, B6, A6 which is the lowest formulation for the one-a-day combinatory pill.

FIG. 5B shows that subpart substrate 148 includes a larger cutout 147 which shows the second grid matrix C1, C12, A12, A7. The next larger grid matrix D1, D18, A18, A13 (tertiary sub-matrix) is printed as indicia on surface 151 of subpart substrate 148. Windows or openings in substrates 144, 146 permit the user-healthcare provider to titrate dosages.

In FIG. 5E, the printed substrate 150 shows the entire matrix I. This permits the doctor to titrate dosages and show these dosages as he titrates the medication through the various grid levels.

The substrates are movable with respect to each other based upon outboard tabs 161, 163, slidably movable in slots 165, 167. Other movable systems permitting movement of the printed substrates having indicia of the subpluralities and formulations can be utilized. The tab in slot slide system in FIG. 5A-5E operates on substrates 144, 146.

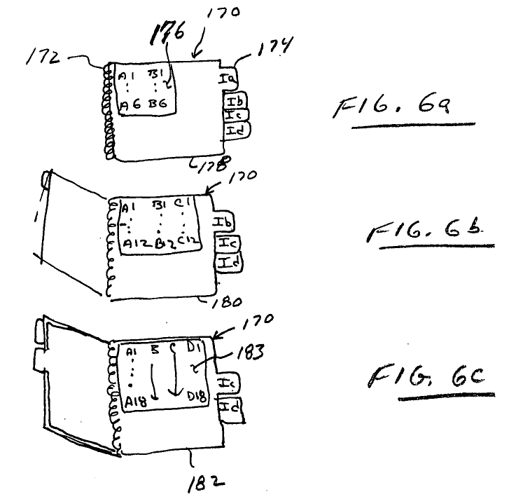

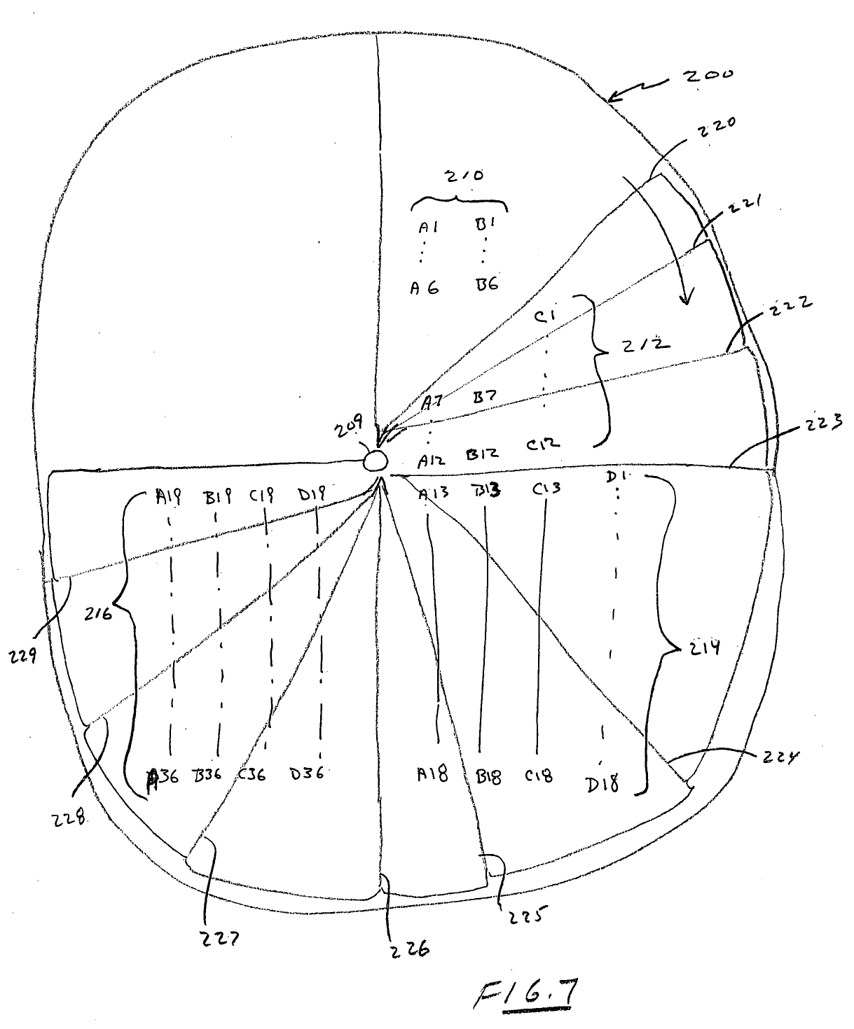

FIGS. 6A-6C show a booklet 170 with hinges or spiral binding 172. These substrates have tabs 174 each marked with indicia representing both the combination pill matrix as well as the subpart of the matrix. Therefore, matrix sub grid Ia is displayed by the use of tab 174 and primary sub grid pattern A1, B1, B6, A6 is shown in window 176 of substrate card 178. With respect to FIG. 6B, substrate card 180 is keyed to secondary sub matrix grid portion Ib which shows grid pattern A1, C1, C12, A12. Card 182 associated with the indicia for tertiary matrix sub-group Ic has a cutout or window 183 permitting the view of sub grid A1, D1, D18, A18. A similar system can be employed for the other matrixes and sub grids.

Selection system shown in FIG. 7 is a generally circular or oval substrate 200 having printed thereon portions of the combination pill matrix I. In region 210, indicia representing the first sub grid A1, B1, B6, A6 is shown. In region 212, the sub grid C1, C12, A12, A7 is shown. In substrate region 214, sub grid A13, D1, D18, A18 is shown. In substrate region 216, pill formulations at subgrid A19, D9, D36, A36 are shown. Movable slides 220-229 cover all or substantially all of these grid indicia. In order to expose substrate portion 210, as well as substrate portion 212, rotatable fan collapsible elements 220, 221, 222 and 223 collapse on top of each other about rotation point 209. In order to expose the sub grid region and printed sub grid portion 214, slidable elements 220-226 are slid and rotated such that they lay adjacent on top of each other. In order to expose the entire grid, the physician or healthcare provider moves slidable elements 226, 227, 228 and 229 thereby exposing all of the matrix I.

The claims appended hereto are meant to cover modifications and changes within the scope and spirit of the present invention.

What is claimed is:

1. A system for selecting one combination one-a-day pill from a plurality of combination pills for treating a patient with a one-a-day, orally administered, pill having a combination of medicaments for hypertension, hypercholesterolemia, hypertriglyceridemia and an anti-platelet medication over a weekly, monthly or quarterly period of time, comprising:
a printed substrate having at least three subparts, each subpart movably disposed with respect to the other subparts;
a first subpart substrate listing indicia representing a first plurality of one-a-day pill formulations consisting essentially of:
a respective first plurality of one-a-day pills each with a therapeutically effective amount of aspirin or clopidogrel bisulfate plus a statin of about 10 mg, a diuretic in a range of about 25 mg to 50 mg, an ace inhibitor in a range of about 10 mg to 40 mg; and a medicament used to lower triglycerides in a range of about 67 mg to 200 mg;
a second subpart substrate listing indicia representing a second plurality of one-a-day pill formulations consisting essentially of:
a respective second plurality of one-a-day pills each with a therapeutically effective amount of aspirin or clopidogrel bisulfate plus a statin in a range of about 10 mg to 20 mg, a diuretic in a range of about 25 mg to 50 mg, an ace inhibitor in a range of about 10 mg to 40 mg; and a medicament used to lower triglycerides in a range of about 67 mg to 200 mg;
a third subpart substrate listing indicia representing a third plurality of one-a-day pill formulations consisting essentially of:
a respective third plurality of one-a-day pills each with a therapeutically effective amount of aspirin or clopidogrel bisulfate plus a statin in a range of about 10 mg to 40 mg, a diuretic in a range of about 25 mg to 50 mg, and an ace inhibitor in a range of about 10 mg to 40 mg;
wherein said respective indicia for said statin, said diuretic, said ace inhibitor, and said medicament used to lower triglycerides represents statin unitized in said ranges and in commonly prescribed dosages; said diuretic unitized in and in commonly prescribed dosages; and said ace inhibitor unitized in said ranges and in commonly prescribed dosages; and said medicament used to lower triglycerides unitized in said ranges and in commonly prescribed dosages;
means for moving said first, second and third subparts independent of each other and facilitating selection of titrated dosages of said combination pills over said weekly, monthly or quarterly period of time.

2. A system for selecting one combination one-a-day pill as claimed in claim 1 wherein said commonly prescribed dosages for said statin being about 10, 20, 40 and 80 mg dosages; said commonly prescribed dosages for said diuretic unitized being about 25 and 50 mg dosages; and said commonly prescribed dosages for said ace inhibitor unitized being about 10, 20 and 40 mg dosages.

3. A system for selecting one combination one-a-day pill as claimed in claim 2 wherein said medicament used to lower triglycerides being a fenofibrate in a range of about 67 mg to 200 mg.

4. A system for selecting one combination one-a-day pill as claimed in claim 3 wherein said commonly prescribed dosages for said fenofibrate being about 67, 160 and 200 mg dosages.

5. A system for selecting one combination one-a-day pill as claimed in claim 1 wherein said indicia representing said first plurality of one-a-day pill formulations is a first color; and said indicia representing said second plurality of one-a-day pill formulations is a second color; and said indicia representing said third plurality of one-a-day pill formulations is a third color, said first, second and third colors begin distinctively different.

6. A system for selecting one combination one-a-day pill as claimed in claim 4 wherein said indicia representing said first plurality of one-a-day pill formulations is a first color; and said indicia representing said second plurality of one-a-day pill formulations is a second color; and said indicia representing said third plurality of one-a-day pill formulations is a third color, said first, second and third colors begin distinctively different.

7. A system for selecting one combination one-a-day pill as claimed in claim 1 wherein said substrate has a fourth subpart substrate listing indicia representing a fourth plurality of one-a-day pill formulations consisting essentially of:
a respective fourth plurality of one-a-day pills a first subgroup of which includes a therapeutically effective amount of aspirin or clopidogrel bisulfate plus a statin in a range of about 10 mg to 80 mg, a diuretic in a range of about 25 mg to 80 mg, an ace inhibitor in a range of about 10 mg to 40 mg and a medicament used to lower triglycerides in a range of about 67 mg to 200 mg; and a second subgroup of which includes a therapeutically effective amount of aspirin or clopidogrel bisulfate plus a statin in a range of about 10 mg to 80 mg, a diuretic in a range of about 25 mg to 80 mg, and an ace inhibitor in a range of about 10 mg to 40 mg.

8. A system for selecting one combination one-a-day pill as claimed in claim 7 wherein said substrate has a fifth subpart substrate listing indicia representing a fifth plurality of one-a-day pill formulations consisting essentially of:
a respective fifth plurality of one-a-day pills each with a therapeutically effective amount of aspirin or clopidogrel bisulfate plus a statin in a range of about 10 mg to 80 mg, an ace inhibitor in a range of about 10 mg to 40 mg and a medicament used to lower triglycerides in a range of about 67 mg to 200 mg;
said means for moving also moving said first, second, third, fourth and fifth subparts independent of each other and facilitating selection of titrated dosages of said combination pills over said weekly, monthly or quarterly period of time.

9. A system for selecting one combination one-a-day pill as claimed in claim 8 wherein said medicament used to lower triglycerides being a fenofibrate in a range of about 67 mg to 200 mg.

10. A system for selecting one combination one-a-day pill as claimed in claim 9 wherein said commonly prescribed dosages for said fenofibrate being about 67, 160 and 200 mg dosages.

11. A system for selecting one combination one-a-day pill as claimed in claim 10 wherein said indicia representing said first plurality of one-a-day pill formulations is a first color; and said indicia representing said second plurality of one-a-day pill formulations is a second color; and said indicia representing said third plurality of one-a-day pill formulations is a third color, said first, second and third colors begin distinctively different.

12. A system for selecting one combination one-a-day pill as claimed in claim 1 wherein said first, second and third subpart substrates are movably coupled together, via said means for moving, by one of hinges, spiral bindings or tabs slidably disposed in slots.

13. A system for selecting one combination one-a-day pill as claimed in claim 11 wherein said first, second, third and fourth subpart substrates are movably coupled together, via said means for moving, by one of hinges, spiral bindings or tabs slidably disposed in slots.

14. A system for selecting one combination pill from a plurality of combination pills for treating a patient with a one-a-day, orally administered, pill having a combination of medicaments for hypertension, hypercholesterolemia, hypertriglyceridemia and an anti-platelet medication over a weekly, monthly or quarterly period of time, comprising:
a printed substrate having at least three subparts, each subpart movably disposed with respect to the other subparts;
a first subpart substrate listing indicia representing a first plurality of one-a-day pill formulations consisting essentially of:
a respective first plurality of one-a-day pills each with a therapeutically effective amount of aspirin or clopidogrel bisulfate plus a statin of about 10 mg, a diuretic in a range of about 25 mg to 50 mg, an ace inhibitor in a range of about 10 mg to 40 mg; and a fenofibrate in a range of about 67 mg to 200 mg;
a second subpart substrate listing indicia representing a second plurality of one-a-day pill formulations consisting essentially of:
a respective second plurality of one-a-day pills each with a therapeutically effective amount of aspirin or clopidogrel bisulfate plus a statin in a range of about 10 mg to 20 mg, a diuretic in a range of about 25 mg to 50 mg, an ace inhibitor in a range of about 10 mg to 40 mg; and a fenofibrate in a range of about 67 mg to 200 mg;
a third subpart substrate listing indicia representing a third plurality of one-a-day pill formulations consisting essentially of:
a respective third plurality of one-a-day pills each with a therapeutically effective amount of aspirin or clopidogrel bisulfate plus a statin in a range of about 10 mg to 40 mg, a diuretic in a range of about 25 mg to 50 mg, and an ace inhibitor in a range of about 10 mg to 40 mg;
wherein said respective indicia for said statin, said diuretic, said ace inhibitor, and fenofibrate represents statin unitized in said ranges of about 10, 20, 40 and 80 mg dosages; said diuretic unitized in said range in about 25 and 50 mg dosages; and said ace inhibitor unitized in said ranges of about 10, 20 and 40 mg dosages, and said fenofibrate unitized in said ranges of about 67, 160 and 200 mg dosages;
means for moving said first, second and third subparts independent of each other and facilitating selection of titrated dosages of said combination pills over said weekly, monthly or quarterly period of time.

15. An information processing system for selecting one combination one-a-day pill from a plurality of combination pills for treating a patient with a one-a-day, orally administered, pill having a combination of medicaments for hypertension, hypercholesterolemia, hypertriglyceridemia and an anti-platelet medication comprising:
a computer having a memory, a display screen and operator input controls, said memory storing at least first, second and third display matrices representing respective first, second and third pluralities of one-a-day pill formulations;
means for displaying, upon a first operator input, indicia from said first matrix representing said first plurality of one-a-day pill formulations consisting essentially of:
a respective first plurality of one-a-day pills each with a therapeutically effective amount of aspirin or clopidogrel bisulfate plus a statin of about 10 mg, a diuretic in a range of about 25 mg to 50 mg, an ace inhibitor in a range of about 10 mg to 40 mg; and a medicament used to lower triglycerides in a range of about 67 mg to 200 mg;
means for displaying, upon a second operator input, indicia from said second matrix representing said second plurality of one-a-day pill formulations consisting essentially of:
a respective second plurality of one-a-day pills each with a therapeutically effective amount of aspirin or clopidogrel bisulfate plus a statin in a range of about 10 mg to 20 mg, a diuretic in a range of about 25 mg to 50 mg, an ace inhibitor in a range of about 10 mg to 40 mg; and a medicament used to lower triglycerides in a range of about 67 mg to 200 mg;
means for displaying, upon a third operator input, indicia from said third matrix representing said third plurality of one-a-day pill formulations consisting essentially of:
a respective third plurality of one-a-day pills each with a therapeutically effective amount of aspirin or clopidogrel bisulfate plus a statin in a range of about 10 mg to 40 mg, a diuretic in a range of about 25 mg to 50 mg, and an ace inhibitor in a range of about 10 mg to 40 mg;
wherein said respective indicia for said statin, said diuretic, said ace inhibitor, and said medicament used to lower triglycerides represents statin unitized in said ranges and in commonly prescribed dosages; said diuretic unitized in said ranges and in commonly prescribed dosages; and said ace inhibitor unitized in said ranges and in commonly prescribed dosages; and said medicament used to lower triglycerides unitized in said ranges and in commonly prescribed dosages; and
an output generator designating a selected one of said one-a-day combination pill upon an operator output selection, said selected one of said one-a-day combination pill being selected from said first, second and third matrices.

16. An information processing system for selecting one combination one-a-day pill as claimed in claim 15 wherein said commonly prescribed dosages for said statin being about 10, 20, 40 and 80 mg dosages;
said commonly prescribed dosages for said diuretic unitized being about 25 and 50 mg dosages; and said commonly prescribed dosages for said ace inhibitor unitized being about 10, 20 and 40 mg dosages.

17. An information processing system for selecting one combination one-a-day pill as claimed in claim 16 wherein said medicament used to lower triglycerides being a fenofibrate in a range of about 67 mg to 200 mg.

18. An information processing system for selecting one combination one-a-day pill as claimed in claim 17 wherein said commonly prescribed dosages for said fenofibrate being about 67, 160 and 200 mg dosages.

19. An information processing system for selecting one combination one-a-day pill as claimed in claim 15 including means for providing a supplemental display listing indicia representing a full formulation of said selected one of said combination one-a-day pill, said selected one of said one-a-day pill being represented in said first, second or third matrices.

20. An information processing system for selecting one combination one-a-day pill as claimed in claim 15 including a dispensing system, said dispensing system including respective storage containers for each formulation of each one-a-day combination pill and a corresponding inventory of one-a-day combination pills therein, a controlled pill dispensing interface accepting, from said output generator, said selected one of said one-a-day combination pill data and dispensing, from the corresponding storage container, a number of said selected one of said one-a-day combination pills.

21. An information processing system for selecting one combination one-a-day pill as claimed in claim 20 wherein said output generator includes a printer outputting a print version of indicia representing said selected one of said one-a-day combination pill and said dispensing system includes a pre-dispensing display showing indicia representing a selected storage container and includes an operator confirmation control which permits said operator to accept or reject said selected storage container dependent upon said print version of indicia representing said selected one of said one-a-day combination pill and said pre-dispensing display.

22. An information processing system for selecting one combination one-a-day pill as claimed in claim 15 wherein said memory stores a fourth matrix representing a fourth plurality of one-a-day pill formulations;
means for displaying, upon a fourth operator input, indicia representing said fourth plurality of one-a-day pill formulations consisting essentially of:
a respective fourth plurality of one-a-day pills a first subgroup of which includes a therapeutically effective amount of aspirin or clopidogrel bisulfate plus a statin in a range of about 10 mg to 80 mg, a diuretic in a range of about 25 mg to 80 mg, an ace inhibitor in a range of about 10 mg to 40 mg and a medicament used to lower triglycerides in a range of about 67 mg to 200 mg; and a second subgroup of which includes a therapeutically effective amount of aspirin or clopidogrel bisulfate plus a statin in a range of about 10 mg to 80 mg, a diuretic in a range of about 25 mg to 80 mg, and an ace inhibitor in a range of about 10 mg to 40 mg.

23. An information processing system for selecting one combination one-a-day pill as claimed in claim 22 wherein said memory stores a fifth matrix representing a fifth plurality of one-a-day pill formulations;
means for displaying, upon a fifth operator input, indicia representing said fifth plurality of one-a-day pill formulations consisting essentially of:
a respective fifth plurality of one-a-day pills each with a therapeutically effective amount of aspirin or clopidogrel bisulfate plus a statin in a range of about 10 mg to 80 mg, an ace inhibitor in a range of about 10 mg to 40 mg and a medicament used to lower triglycerides in a range of about 67 mg to 200 mg.

24. An information processing system for selecting one combination one-a-day pill as claimed in claim 23 wherein said medicament used to lower triglycerides being a fenofibrate in a range of about 67 mg to 200 mg.

25. An information processing system for selecting one combination one-a-day pill as claimed in claim 24 wherein said commonly prescribed dosages for said fenofibrate being about 67, 160 and 200 mg dosages.

26. An information processing system for selecting one combination one-a-day pill as claimed in claim 25 wherein said output generator lists said selected one of said one-a-day combination pill from said first, second, third, fourth and fifth matrices.

27. A dispensing system for selecting one combination one-a-day pill from a plurality of combination pills for treating a patient with a one-a-day, orally administered, pill having a combination of medicaments for hypertension, hypercholesterolemia, hypertriglyceridemia and an anti-platelet medication comprising:
a dispensing system, said dispensing system including respective storage containers for each formulation of each one-a-day combination pill and a corresponding inventory of one-a-day combination pills therein,
said respective storage containers having first, second and third pluralities of one-a-day pill formulations;
said first plurality of one-a-day pill formulations consisting essentially of one-a-day pills each with a therapeutically effective amount of aspirin or clopidogrel bisulfate plus a statin of about 10 mg, a diuretic in a range of about 25 mg to 50 mg, an ace inhibitor in a range of about 10 mg to 40 mg; and a medicament used to lower triglycerides in a range of about 67 mg to 200 mg;
said second plurality of one-a-day pill formulations consisting essentially of one-a-day pills each with a therapeutically effective amount of aspirin or clopidogrel bisulfate plus a statin in a range of about 10 mg to 20 mg, a diuretic in a range of about 25 mg to 50 mg, an ace inhibitor in a range of about 10 mg to 40 mg; and a medicament used to lower triglycerides in a range of about 67 mg to 200 mg;
said third plurality of one-a-day pill formulations consisting essentially of one-a-day pills each with a therapeutically effective amount of aspirin or clopidogrel bisulfate plus a statin in a range of about 10 mg to 40 mg, a diuretic in a range of about 25 mg to 50 mg, and an ace inhibitor in a range of about 10 mg to 40 mg;
wherein said statin, diuretic, ace inhibitor, and medicament used to lower triglycerides represents are unitized such that said statin is unitized in said ranges and in commonly prescribed dosages; said diuretic is unitized in said ranges and in commonly prescribed dosages; and said ace inhibitor is unitized in said ranges and in commonly prescribed dosages; and said medicament used to lower triglycerides is unitized in said ranges and in commonly prescribed dosages; and
a controlled pill dispensing interface accepting a data input for a selected one of said one-a-day combination pill and dispensing, from the corresponding storage container, a number of said selected one of said one-a-day combination pills.

28. A dispensing system for selecting one combination one-a-day pill as claimed in claim 27 wherein said commonly prescribed dosages for said statin is about 10, 20, 40 and 80 mg dosages; said commonly prescribed dosages for said diuretic unitized is about 25 and 50 mg dosages; and said commonly prescribed dosages for said ace inhibitor unitized is about 10, 20 and 40 mg dosages.

29. A dispensing system for selecting one combination one-a-day pill as claimed in claim 28 wherein said medicament used to lower triglycerides is a fenofibrate in a range of about 67 mg to 200 mg.

30. A dispensing system for selecting one combination one-a-day pill as claimed in claim 29 wherein said commonly prescribed dosages for said fenofibrate is about 67, 160 and 200 mg dosages.

31. A dispensing system for selecting one combination one-a-day pill as claimed in claim 27 including means for providing a display listing indicia representing said selected one of said combination one-a-day pill.

32. A dispensing system for selecting one combination one-a-day pill as claimed in claim 27 including a pill bottle, each said storage container having a pill dispensing port thereon, and said controlled pill dispensing interface including:
means to position said pill bottle relative to the storage container for said selected one of said one-a-day combination pill;
means to controllably open and close a corresponding pill dispensing port for said storage container for said selected one of said one-a-day combination pill thereby to release said number of said selected one of said one-a-day combination pill into said pill bottle.

33. A dispensing system for selecting one combination one-a-day pill as claimed in claim 32 including a pre-dispensing display showing indicia representing said selected storage container and including an operator confirmation control which permits said operator to accept or reject said selected storage container dependent upon script instructions for said patient.

34. A dispensing system for selecting one combination one-a-day pill as claimed in claim 32 including a label generator for printing indicia representing said selected one of said one-a-day combination pill.

35. A dispensing system for selecting one combination one-a-day pill as claimed in claim 27 including respective storage containers for a fourth plurality of one-a-day pill formulations;
said fourth plurality of one-a-day pill formulations consisting essentially of a first subgroup of combination pills which includes a therapeutically effective amount of aspirin or clopidogrel bisulfate plus a statin in a range of about 10 mg to 80 mg, a diuretic in a range of about 25 mg to 80 mg, an ace inhibitor in a range of about 10 mg to 40 mg and a medicament used to lower triglycerides in a range of about 67 mg to 200 mg; and a second subgroup of combination pills which includes a therapeutically effective amount of aspirin or clopidogrel bisulfate plus a statin in a range of about 10 mg to 80 mg, a diuretic in a range of about 25 mg to 80 mg, and an ace inhibitor in a range of about 10 mg to 40 mg.

36. A dispensing system for selecting one combination one-a-day pill as claimed in claim 35 including respective storage containers for a fifth plurality of one-a-day pill formulations;
said fifth plurality of one-a-day pill formulations consisting essentially of one-a-day pills each with a therapeutically effective amount of aspirin or clopidogrel bisulfate plus a statin in a range of about 10 mg to 80 mg, an ace inhibitor in a range of about 10 mg to 40 mg and a medicament used to lower triglycerides in a range of about 67 mg to 200 mg.

37. A dispensing system for selecting one combination one-a-day pill as claimed in claim 36 wherein said medicament used to lower triglycerides being a fenofibrate in a range of about 67 mg to 200 mg.

38. A dispensing system for selecting one combination one-a-day pill as claimed in claim 37 wherein said commonly prescribed dosages for said fenofibrate being about 67, 160 and 200 mg dosages.

39. A process to mitigate coronary heart disease, cardiovascular disease or diabetes mellitus with a one-a-day, orally administered, pill selected from a matrix, the process comprising:
selecting a one-a-day pharmaceutical combination from a plurality of one-a-day pill formulations listed on a substrate in a matrix form, said matrix listing:
a plurality of one-a-day pill formulations, each one-a-day pill formulation including a therapeutically effective amount of aspirin or clopidogrel bisulfate plus a statin in a range of 10 mg to 80 mg, a diuretic in a range of 25 mg to 80 mg, and an ace inhibitor in a range of 10 mg to 40 mg;
wherein said statin is unitized in said range in about 10, 20, 40 and 80 mg dosages; said diuretic is unitized in said range in about 25, 50 and 80 mg dosages; and said ace inhibitor is unitized in said range in about 10, 20 and 40 mg dosages.

40. A process as claimed in claim 39 wherein said plurality of one-a-day pill formulations listed on said matrix includes:
a further plurality of one-a-day pills, each one of said further plurality of one-a-day pill formulations including a therapeutically effective amount of aspirin or clopidogrel bisulfate plus a statin in a range of 10 mg to 20 mg, a diuretic in a range of 25 mg to 50 mg, an ace inhibitor in a range of 10 mg to 40 mg and a fenofibrate in a range of about 67 mg to 200 mg; and
wherein said fenofibrate is unitized in about 67, 160 and 200 mg dosages.

41. A process as claimed in claim 39 wherein said one-a-day pill formulations listed in said matrix includes:
one-a-day pill formulations with a therapeutically effective amount of aspirin or clopidogrel bisulfate plus a statin in a range of 10 mg to 80 mg, an ace inhibitor in a range of 10 mg to 40 mg and a fenofibrate in a range of about 67 mg to 200 mg; and
wherein said fenofibrate is unitized in about 67, 160 and 200 mg dosages.

42. A process to mitigate coronary heart disease, cardiovascular disease or diabetes mellitus with a one-a-day, orally administered, pill selected from a matrix, comprising:
selecting a one-a-day pill formulation from a plurality of formulations listed on a substrate in a matrix form, said matrix listing:
a plurality of one-a-day pill formulations each with a therapeutically effective amount of aspirin or clopidogrel bisulfate plus a statin in a range of 10 mg to 40 mg, a diuretic in a range of 25 mg to 50 mg, and an ace inhibitor in a range of 10 mg to 40 mg;
a further plurality of one-a-day pill formulations each with a therapeutically effective amount of aspirin or clopidogrel bisulfate plus a statin in a range of 10 mg to 20 mg, a diuretic in a range of 25 mg to 50 mg, an ace inhibitor in a range of 10 mg to 40 mg and a fenofibrate in a range of about 67 mg to 200 mg;
wherein said statins are unitized in said range in about 10 and 20 mg dosages; said diuretic is unitized in said range in about 25 and 50 mg dosages; said ace inhibitor is unitized in said range in about 10, 20 and 40 mg dosages; and said fenofibrate is unitized in said range in about 67, 160 and 200 mg dosages.

43. A process to mitigate coronary heart disease, cardiovascular disease or diabetes mellitus with a one-a-day, orally administered, pill selected from a matrix, comprising:
selecting a one-a-day pill formulation from a plurality of formulations using representative indicia listed on a substrate in a matrix form, said matrix listing:
a plurality of one-a-day pill formulations, each one-a-day pill with a therapeutically effective amount of aspirin or clopidogrel bisulfate plus a statin in a range of 10 mg to 80 mg, a diuretic in a range of 25 mg to 80 mg, and an ace inhibitor in a range of 10 mg to 40 mg;
a further plurality of one-a-day pill formulations, each one-a-day pill with a therapeutically effective amount of aspirin or clopidogrel bisulfate plus a statin in a range of 10 mg to 80 mg, an ace inhibitor in a range of 10 mg to 40 mg and a medicament used to lower triglycerides in a range of about 67 mg to 200 mg;
wherein said statin is unitized in said range in about 10, 20, 40 and 80 mg dosages; said diuretic is unitized in said range in about 25, 50 and 80 mg dosages; said ace inhibitor is unitized in said range in about 10, 20 and 40 mg dosages; and said medicament used to lower triglycerides is unitized in said range in about 67, 160 and 200 mg dosages.

44. A process to mitigate coronary heart disease, cardiovascular disease or diabetes mellitus with a one-a-day, orally administered, pill selected from a matrix, comprising:
selecting a one-a-day pill formulation from a plurality of formulations listed on a substrate in a matrix form, said matrix listing:
a plurality of one-a-day pill formulations, each one-a-day pill with a therapeutically effective amount of aspirin or clopidogrel bisulfate plus a statin in a range of 10 mg to 80 mg, a diuretic in a range of 25 mg to 80 mg, and an ace inhibitor in a range of 10 mg to 40 mg;
a further plurality of one-a-day pill formulations, each one-a-day pill with a therapeutically effective amount of aspirin or clopidogrel bisulfate plus a statin in a range of 10 mg to 20 mg, a diuretic in a range of 25 mg to 50 mg, an ace inhibitor in a range of 10 mg to 40 mg and a fenofibrate in a range of about 67 mg to 200 mg;
an additional plurality of one-a-day pill formulations, each one-a-day pill with a therapeutically effective amount of aspirin or clopidogrel bisulfate plus a statin in a range of 10 mg to 80 mg, an ace inhibitor in a range of 10 mg to 40 mg and a fenofibrate in a range of about 67 mg to 200 mg;
wherein said statin is unitized in said range in about 10, 20, 40 and 80 mg dosages; said diuretic is unitized in said range in about 25, 50 and 80 mg dosages; said ace inhibitor is unitized in said range in about 10, 20 and 40 mg dosages; and said fenofibrate is unitized in said range in about 67, 160 and 200 mg dosages.

45. A process to mitigate coronary heart disease, cardiovascular disease or diabetes mellitus with a one-a-day, orally administered, pill selected from a matrix, comprising:
selecting a one-a-day pill formulation from a plurality of formulations listed on a substrate in a matrix form, said matrix listing:
a first plurality of one-a-day pills with a therapeutically effective amount of aspirin or clopidogrel bisulfate plus a statin in unitized dosages a range of 10 mg to 80 mg, a diuretic in unitized dosages in a range of 25 mg to 80 mg, and an ace inhibitor in unitized dosages in a range of 10 mg to 40 mg;
a second plurality of one-a-day pills with a therapeutically effective amount of aspirin or clopidogrel bisulfate plus a statin in unitized dosages in a range of 10 mg to 20 mg, a diuretic in unitized dosages in a range of 25 mg to 50 mg, an ace inhibitor in unitized dosages in a range of 10 mg to 40 mg and a fenofibrate in unitized dosages in a range of about 67 mg to 200 mg;
a third plurality of one-a-day pills with a therapeutically effective amount of aspirin or clopidogrel bisulfate plus a statin in unitized dosages in a range of 10 mg to 80 mg, an ace inhibitor in unitized dosages in a range of 10 mg to 40 mg and a fenofibrate in unitized dosages in a range of about 67 mg to 200 mg;
a fourth plurality one-a-day pills with a therapeutically effective amount of aspirin or clopidogrel bisulfate plus a diuretic in unitized dosages in a range of 25 mg to 80 mg, and an ace inhibitor in unitized dosages in a range of 10 mg to 40 mg;
a fifth plurality of one-a-day pills with a therapeutically effective amount of aspirin or clopidogrel bisulfate plus a diuretic in unitized dosages in a range of 25 mg to 80 mg, an ace inhibitor in unitized dosages in a range of 10 mg to 40 mg and a fenofibrate in unitized dosages in a range of about 160 mg to 200 mg;
a sixth plurality of one-a-day pills with a therapeutically effective amount of aspirin or clopidogrel bisulfate plus an ace inhibitor in unitized dosages in a range of 10 mg to 40 mg and a fenofibrate in unitized dosages in a range of about 160 mg to 200 mg;
a seventh plurality of one-a-day pills with a therapeutically effective amount of aspirin or clopidogrel bisulfate plus a statin in unitized dosages in a range of 10 mg to 80 mg, and an ace inhibitor in unitized dosages in a range of 10 mg to 40 mg;
an eighth plurality of one-a-day pills with a therapeutically effective amount of aspirin or clopidogrel bisulfate plus a statin in unitized dosages in a range of 10 mg to 80 mg and a diuretic in unitized dosages in a range of 25 mg to 80 mg;
a ninth plurality of one-a-day pills with a therapeutically effective amount of aspirin or clopidogrel bisulfate plus a statin in unitized dosages in a range of 10 mg to 80 mg, and a fenofibrate in unitized dosages in a range of about 67 mg to 200 mg;
a tenth plurality of one-a-day pills with a therapeutically effective amount of aspirin or clopidogrel bisulfate plus a statin in unitized dosages in a range of 10 mg to 80 mg, a diuretic in unitized dosages in a range of 25 mg to 80 mg, and a fenofibrate in unitized dosages in a range of about 67 mg to 200 mg;
an eleventh plurality of one-a-day pills with a therapeutically effective amount of aspirin or clopidogrel bisulfate plus a diuretic in unitized dosages in a range of 25 mg to 50 mg and a fenofibrate in unitized dosages in a range of about 67 mg to 200 mg;
wherein said statins are unitized in 10, 20, 40 and 80 mg dosages; said diuretic is unitized in 25, 50 and 80 mg dosages; said ace inhibitor is unitized in 10, 20 and 40 mg dosages; and said fenofibrate is unitized in about 67, 160 and 200 mg dosages.
